(19) United States
(12) Patent Application Publication
(10) Pub. No.: US 2010/0029700 A1
(43) Pub. Date: Feb. 4, 2010

(54) 1-(2-ARYL-2-OXOETHYL)-3-PHENYL-1, 4-DIAZASPIRO [4.5]DEC-3-EN-2-ONE DERIVATIVES AND THEIR USE AS GLYCINE TRANSPORTER INHIBITORS

(76) Inventors: Clive Leslie Branch, Essex (GB); Howard Marshall, Essex (GB); David John Nash, Essex (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/296,712

(22) PCT Filed: Apr. 10, 2007
(86) PCT No.: PCT/EP2007/053468

§ 371 Date: Sep. 24, 2009

(30) Foreign Application Priority Data
Apr. 12, 2006 (GB) 0607398.5

(51) Int. Cl. A61K 31/438; C07D 209/54; A61K 31/415; C07D 401/02

(52) U.S. Cl. 514/278; 548/408; 514/385; 546/15

(57) ABSTRACT

Novel acetophenone compounds of formula (I), compositions containing (I), processes for the preparation of (I), and use of (I) as inhibitors of the glycine transporter, are provided.

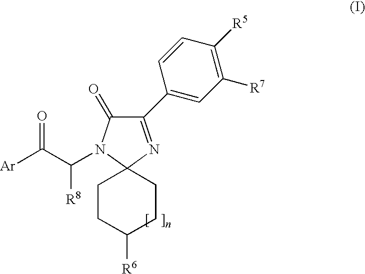

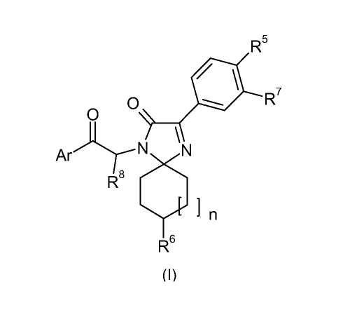

Definitions of Ar, R⁵, R⁶, R⁷, R⁸ and n are provided in the specification.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-1a, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995 : 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Enql. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states. Glycine transport inhibitors are already known in the art, for example as disclosed in published international patent application WO03/055478 (SmithKline Beecham).

It has now been found that a novel class of compounds inhibit GlyT1 transporters and are thus useful in the treatment of certain neurological and neuropsychiatric disorders, including schizophrenia.

Thus, in the first aspect, there is provided a compound of formula (I) or a salt or solvate thereof:

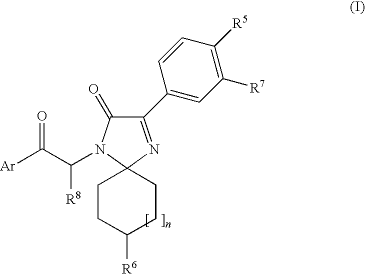

wherein:

Ar is selected from naphthyl optionally substituted with one or more groups Y, pyridinyl optionally substituted with one or more groups Y, and the group

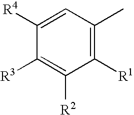

Y is selected from C₁-C₄alkyl, C₁-C₄alkoxy, halo, haloC₁-C₄alkyl, haloC₁-C₄alkoxy, and cyano;
R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ (where R⁹ and R are independently selected from H and C₁-C₄alkyl), heteroaryl and cyano;
R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ (where R⁹ and R¹⁰ are independently selected from H and C₁-C₄alkyl), heteroaryl and cyano;
R³ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ (where R⁹ and R¹⁰ are independently selected from H and C₁-C₄alkyl), heteroaryl and cyano;
or R² and R³ together form a group selected from —O—CH₂—O— and —O—CH₂-CH₂—O—;

R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ (where R⁹ and R¹⁰ are independently selected from H and C₁-C₄alkyl), heteroaryl and cyano;

R⁵ is selected from H, C₁-C₄alkyl, C₁-C₄alkoxy, halo, haloC₁-C₄alkyl, haloC₁-C₄alkoxy, C₁-C₄alkoxyC₁-C₄alkyl, C₁-C₄alkoxyC₁-C₄alkoxy, and cyano;
R⁷ is selected from H, C₁-C₄alkyl, C₁-C₄alkoxy, halo, haloC₁-C₄alkyl, haloC₁-C₄alkoxy, C₁-C₄alkoxyC₁-C₄alkyl, C₁-C₄alkoxyC₁-C₄alkoxy, and cyano;
R⁶ is selected from H and methyl;
R⁸ is selected from H and methyl; and
n is selected from 0, 1 and 2.

Compounds of formula (I) have been found to inhibit GlyT1 transporters.

In an embodiment, Ar is selected from naphthyl, pyridinyl and the group

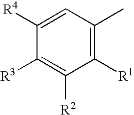

In a further embodiment, Ar is a group

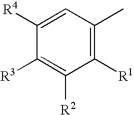

In one embodiment, R¹ is selected from H, C₁-C₂alkyl, C₁-C₂alkoxy, halo, haloC₁-C₂alkyl, haloC₁-C₂alkoxy, heteroaryl and cyano; in a further embodiment, R¹ is selected from H, methyl, methoxy, halo, trifluoromethyl, pyridinyl and cyano. In a further embodiment R¹ is selected from H, methyl, methoxy, pyridin-2-yl and trifluoromethyl. For example, halo groups may be selected from bromo, chloro and fluoro; particularly chloro and fluoro.

In one embodiment, R² is selected from H, C₁-C₂alkyl, C₁-C₂alkoxy, halo, haloC₁-C₂alkyl, haloC₁-C₂alkoxy, heteroaryl and cyano; in a further embodiment, R² is selected from H, methyl, methoxy, halo, trifluoromethyl, pyridinyl and cyano. In a further embodiment, R² is selected from H, methyl, methoxy, halo, pyridin-2-yl and trifluoromethyl. For example, halo groups may be selected from bromo, chloro and fluoro; particularly chloro and fluoro, for example chloro.

In one embodiment, R³ is selected from H, C₁-C₂alkyl, C₁-C₂alkoxy, halo, haloC₁-C₂alkyl, haloC₁-C₂alkoxy, heteroaryl and cyano; in a further embodiment, R³ is selected from H, methyl, methoxy, halo, trifluoromethyl, pyridinyl and cyano. In a further embodiment, R³ is selected from H, methyl, methoxy, halo, pyridin-2-yl and trifluoromethyl. For example, halo groups are selected from bromo, chloro and fluoro; particularly chloro and fluoro, for example chloro.

In an alternative embodiment, R² and R³ together form a group selected from —O—CH₂—O— and —O—CH₂—CH₂—O—.

In one embodiment, R⁴ is selected from H, C₁-C₂alkyl, C₁-C₂alkoxy, halo, haloC₁-C₂alkyl, haloC₁-C₂alkoxy, heteroaryl and cyano; in a further embodiment, R⁴ is selected from H, methyl, methoxy, halo, trifluoromethyl, pyridinyl and cyano. In a further embodiment, R⁴ is selected from H, methyl, methoxy, halo, pyridin-2-yl and trifluoromethyl. For example, halo groups are selected from bromo, chloro and fluoro; particularly chloro and fluoro, for example fluoro. In a further embodiment, R⁴ is H.

In one embodiment, R⁵ is selected from H, C₁-C₂alkyl, C₁-C₂alkoxy, halo, haloC₁-C₂alkyl, haloC₁-C₂alkoxy, C₁-C₂alkoxyC₁-C₂alkyl, C₁-C₂alkoxyC₁-C₂alkoxy and cyano; in a further embodiment, R⁵ is selected from H, C₁-C₂alkyl, C₁-C₂alkoxy, halo, haloC₁-C₂alkyl and cyano. In a further embodiment, R⁵ is selected from H, methyl, methoxy, halo, trifluoromethyl and cyano. For example, halo groups are selected from bromo, chloro and fluoro; particularly chloro and fluoro, for example chloro.

In one embodiment, R⁷ is selected from H, C₁-C₂alkyl, C₁-C₂alkoxy, halo, haloC₁-C₂alkyl, haloC₁-C₂alkoxy, C₁-C₂alkoxyC₁-C₂alkyl, C₁-C₂alkoxyC₁-C₂alkoxy and cyano; in a further embodiment, R⁷ is selected from H, C₁-C₂alkyl, C₁-C₂alkoxy, halo, and haloC₁-C₂alkyl. In a further embodiment, R⁷ is H.

In one embodiment, R⁶ is H.

In one embodiment, R⁸ is H. In an alternative embodiment, R⁸ is methyl.

In one embodiment, n is selected from 0 and 1. In a further embodiment, n is 1. In an alternative embodiment, n is 0.

In one embodiment:

Ar is selected from naphthyl, pyridinyl and the group

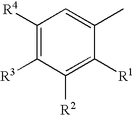

R¹ is selected from H, C₁-C₂alkyl, C₁-C₂alkoxy, halo, haloC₁-C₂alkyl, haloC₁-C₂alkoxy, heteroaryl, and cyano;
R² is selected from H, C₁-C₂alkyl, C₁-C₂alkoxy, halo, haloC₁-C₂alkyl, haloC₁-C₂alkoxy, heteroaryl, and cyano;
R³ is selected from H, C₁-C₂alkyl, C₁-C₂alkoxy, halo, haloC₁-C₂alkyl, haloC₁-C₂alkoxy, heteroaryl, and cyano;
or R² and R³ together form a group selected from —O—CH₂—O— and —O—CH₂-CH₂—O—;
R⁴ is selected from H, C₁-C₂alkyl, C₁-C₂alkoxy, halo, haloC₁-C₂alkyl, haloC₁-C₂alkoxy, heteroaryl, and cyano;
R⁵ is selected from H, C₁-C₂alkyl, C₁-C₂alkoxy, halo, haloC₁-C₂alkyl, haloC₁-C₂alkoxy, C₁-C₂alkoxyC₁-C₂alkyl, C₁-C₂alkoxyC₁-C₂alkoxy, and cyano;
R⁷ is selected from H, C₁-C₂alkyl, C₁-C₂alkoxy, halo, haloC₁-C₂alkyl, haloC₁-C₂alkoxy, C₁-C₂alkoxyC₁-C₂alkyl, C₁-C₂alkoxyC₁-C₂alkoxy, and cyano;

### R ⁶is H;

R⁸ is selected from H and methyl; and
n is selected from 0 and 1.

In one embodiment:

Ar is selected from naphthyl and the group

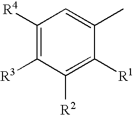

R¹ is selected from H, methyl, methoxy, halo, trifluoromethyl, pyridinyl, and cyano;
R² is selected from H, methyl, methoxy, halo, trifluoromethyl, pyridinyl, and cyano;
R³ is selected from H, C₁-C₂alkyl, C₁-C₂alkoxy, halo, haloC₁-C₂alkyl, haloC₁-C₂alkoxy, pyridinyl, and cyano;
or R² and R³ together form a group selected from —O—CH₂—O— and —O—CH₂-CH₂—O—;
R⁴ is selected from H, methyl, methoxy, halo, trifluoromethyl, pyridinyl, and cyano;
R⁵ is selected from H, C₁-C₂alkyl, C₁-C₂alkoxy, halo, haloC₁-C₂alkyl, and cyano;
R⁷ is selected from H, C₁-C₂alkyl, C₁-C₂alkoxy, halo, and haloC₁-C₂alkyl;

### R ⁶is H;

R⁸ is selected from H and methyl; and
n is selected from 0 and 1

In one embodiment:

Ar is selected from naphthyl and the group

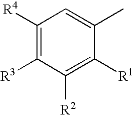

R¹ is selected from H, methyl, methoxy, chloro, fluoro, trifluoromethyl, pyridinyl, and cyano;
R² is selected from H, methyl, methoxy, bromo, chloro, fluoro, trifluoromethyl, pyridinyl, and cyano;
R³ is selected from H, C₁-C₂alkyl, C₁-C₂alkoxy, chloro, fluoro, chloroC₁-C₂alkyl, fluoroC₁-C₂alkyl, chloroC₁-C₂alkoxy, fluoroC₁-C₂alkoxy, pyridinyl, and cyano; or R² and R³ together form a group selected from —O—CH₂—O— and —O—CH₂-CH₂—O—;
R⁴ is selected from H, methyl, methoxy, bromo, chloro, fluoro, trifluoromethyl, pyridinyl, and cyano;
R⁵ is selected from H, methyl, methoxy, halo, trifluoromethyl and cyano;
R⁷ is selected from H, C₁-C₂alkyl, C₁-C₂alkoxy, halo, and haloC₁-C₂alkyl;

### R⁶ is H;

R⁸ is selected from H and methyl; and
n is selected from 0 and 1

In one embodiment, there is provided a compound of formula (IA) or a salt or solvate thereof:

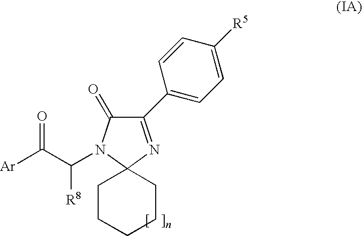

wherein:

Ar is selected from naphthyl and the group

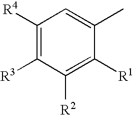

R¹ is selected from hydrogen, methyl, chloro, fluoro, methoxy and trifluoromethyl;
R² is selected from hydrogen, methyl, trifluoromethyl, fluoro, chloro, bromo, methoxy, cyano and pyridin-2-yl;
R³ is selected from hydrogen, methyl, trifluoromethyl, methoxy, chloro, fluoro and cyano; or R² and R³ together form —O—CH₂-CH₂—O—;
R⁴ is selected from hydrogen, methyl, trifluoromethyl, fluoro, chloro, bromo, methoxy, cyano and pyridin-2-yl;
R⁵ is selected from chloro, trifluoromethyl, methoxy and cyano;
R⁸ is selected from hydrogen and methyl; and
n is 0 or 1.

For the avoidance of doubt, the embodiments of any one feature of the compounds of the invention may be combined with any embodiment of another feature of compounds of the invention to create a further embodiment.

The terms ‘C₁-C₂alkyl’ or ‘C₁-C₄alkyl’ as used herein refer to a linear or branched alkyl group containing from 1 to 2 or 1 to 4 carbon atoms respectively. Examples of C₁-C₂alkyl groups include methyl and ethyl. Examples of C₁-C₄alkyl groups include, in addition to the above, n-propyl, isopropyl, n-butyl, iso-butyl, sec-butyl and tert butyl.

The terms ‘C₁-C₂alkoxy’ or ‘C₁-C₄alkoxy’ as used herein refer to a linear or branched chain alkoxy group containing from 1 to 2 or 1 to 4 carbon atoms respectively. Examples of C₁-C₂alkoxy groups include methoxy and ethoxy. Examples of C₁-C₄alkoxy groups include, in addition to the above, propoxy, prop-2-oxy, butoxy, but-2-oxy and 2-methyl-prop-2-oxy.

The term ‘halo’ or ‘halogen’ as used herein refers to a fluorine, chlorine, bromine or iodine atom.

The terms ‘haloC₁-C₂alkyl’, or ‘haloC₁-C₄alkyl’ as used herein refer to an alkyl group as defined above wherein at least one hydrogen atom in the alkyl group is replaced with halogen. Examples include trifluoromethyl and the like.

The terms ‘haloC₁-C₂alkoxy’ and ‘haloC₁-C₄alkoxy’ as used herein refer to an alkoxy group as defined above wherein at least one hydrogen atom in the alkyl group is replaced with halogen. Examples include —OCHF₂, —OCF₃, and the like

The term ‘C₃-C₆cycloalkyl’ as used herein refers to a non aromatic monocyclic hydrocarbon ring of 3 to 6 carbon atom such as, for example, cyclopropyl, cyclobutyl, cyclopentyl, and cyclohexyl

The terms ‘C₁-C₂alkylthio’ or ‘C₁-C₄alkylthio’ as used herein refer to a linear or branched alkylthio group containing from 1 to 2 or 1 to 4 carbon atoms respectively. Examples of C₁-C₂alkylthio groups include methylthio and ethylthio. Examples of C₁-C₄alkylthio groups include, in addition to the above, propylthio, propyl-2-thio, butylthio, butyl-2-thio and 2-methyl-propyl-2-thio.

The terms ‘C₁-C₂alkylsulfonyl’ or ‘C₁-C₄alkylsulfonyl’ as used herein refer to a linear or branched alkylsulfonyl group containing from 1 to 2 or 1 to 4 carbon atoms respectively. Examples of C₁-C₂alkylsulfonyl groups include methylsulfonyl and ethylsulfonyl. Examples of C₁-C₄alkylsulfonyl groups include, in addition to the above, propylsulfonyl, propyl-2-sulfonyl, butylsulfonyl, butyl-2-sulfonyl and 2-methyl-propyl-2-sulfonyl.

The terms ‘C₁-C₂alkoxyC₁-C₂alkyl’ and ‘C₁-C₄alkoxyC₁-C₄alkyl’ as used herein refer to a linear or branched alkoxyalkyl group wherein ‘alkoxy’ and ‘alkyl’ are as defined above. Examples of C₁-C₂alkoxyC₁-C₂alkyl groups include methoxymethy, ethoxymethyl, methoxyethyl and ethoxyethyl. Examples of C₁-C₄alkoxyC₁-C₄alkyl groups include, in addition to the above, propoxyethyl, ethoxypropyl and the like.

The terms ‘C₁-C₂alkoxyC₁-C₂alkoxy’ and ‘C₁-C₄alkoxyC₁-C₄alkoxy’ as used herein refer to a linear or branched alkoxyalkoxy group wherein ‘alkoxy’ is as defined above. Examples of C₁-C₂alkoxyC₁-C₂alkoxy groups include methoxymethoxy, methoxyethoxy, ethoxymethoxy and ethoxyethoxy. Examples of C₁-C₄alkoxyC₁-C₄alkoxy groups include, in addition to the above, propoxyethoxy, ethoxypropoxy and the like.

The term ‘heteroaryl’ as used herein refers to a 5- or 6-membered aromatic heterocyclic ring containing 1-3 heteroatoms independently selected from N, O or S. Examples include pyridyl, thienyl, furanyl, thiazolyl, oxazolyl and the like.

As used herein, the term “salt” refers to any salt of a compound according to the present invention prepared from an inorganic or organic acid. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1S)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic and p-toluenesulfonic, acids. Salts having a non-pharmaceutically acceptable anion or cation are within the scope of the invention as useful intermediates for the preparation of salts and/or for use in non-therapeutic, for example, in vitro, situations. The salts may have any suitable stoichiometry. For example, a salt may have 1:1 or 2:1 stoichiometry. Non-integral stoichiometry ratios are also possible.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. In one embodiment, the solvent used is a pharmaceutically acceptable solvent. Examples of suitable pharmaceutically acceptable solvents include water, ethanol and acetic acid. In one embodiment the solvent used is water.

Examples of compounds of the invention include:

1-[2-(4-chloro-3-methylphenyl)-2-oxoethyl]-3-(4-chlorophenyl)-1,4-diazaspiro[4.5]dec-3-en-2-one;
3-(4-chlorophenyl)-1-{2-oxo-2-[3-(trifluoromethyl)phenyl]ethyl}-1,4-diazaspiro[4.5]dec-3-en-2-one;
1-[2-(3,4-difluorophenyl)-2-oxoethyl]-3-[4-(trifluoromethyl)phenyl]-1,4-diazaspiro[4.5]dec-3-en-2-one;
1-[2-(4-chloro-3-methylphenyl)-2-oxoethyl]-3-[4-(methyloxy)phenyl]-1,4-diazaspiro[4.5]dec-3-en-2-one;
3-(4-chlorophenyl)-1-{2-[2-(methyloxy)phenyl]-2-oxoethyl}- 1,4-diazaspiro[4.5]dec-3-en-2-one;
3-(4-chlorophenyl)-1-(2-oxo-2-phenylethyl)-1,4-diazaspiro[4.5]dec-3-en-2-one;
4-{[3-(4-chlorophenyl)-2-oxo-1,4-diazaspiro[4.5]dec-3-en-1-yl]acetyl}benzonitrile;
3-(4-chlorophenyl)-1-[2-(2-naphthalenyl)-2-oxoethyl]-1,4-diazaspiro[4.5]dec-3-en-2-one;
3-(4-chlorophenyl)-1-{2-oxo-2-[4-(trifluoromethyl)phenyl]ethyl}-1,4-diazaspiro[4.5]dec-3-en-2-one;
3-(4-chlorophenyl)-1-[2-(2,4-dimethylphenyl)-2-oxoethyl]-1,4-diazaspiro[4.5]dec-3-en-2-one;
1-[2-(4-chloro-3-methylphenyl)-2-oxoethyl]-3-[4-(trifluoromethyl)phenyl]-1,4-diazaspiro[4.5]dec-3-en-2-one;
3-(4-chlorophenyl)-1-[2-(3,4-difluorophenyl)-2-oxoethyl]-1,4-diazaspiro[4.5]dec-3-en-2-one;
3-(4-chlorophenyl)-1-[2-(2,3-dihydro-1,4-benzodioxin-6-yl)-2-oxoethyl]-1,4-diazaspiro[4.5]dec-3-en-2-one;
3-(4-chlorophenyl)-1-{2-[3-(methyloxy)phenyl]-2-oxoethyl}-1,4-diazaspiro[4.5]dec-3-en-2-one;
3-(4-chlorophenyl)-1-{2-[4-( methyloxy)phenyl]-2-oxoethyl}-1,4-diazaspiro[4.5]dec-3-en-2-one;
3-(4-chlorophenyl)-1-{2-oxo-2-[2-(trifluoromethyl)phenyl]ethyl}-1,4-diazaspiro[4.5]dec-3-en-2-one; and
3-(4-chlorophenyl)-1-(1-methyl-2-oxo-2-phenylethyl)-1,4-diazaspiro[4.5]dec-3-en-2-one and salts and solvates thereof.

Further examples include:

3-(4-chlorophenyl)-1-[2-(4-chlorophenyl)-2-oxoethyl]-1,4-diazaspiro[4.5]dec-3-en-2-one;
3-(4-chlorophenyl)-1-{2-[5-fluoro-2-(methyloxy)phenyl]-2-oxoethyl}-1,4-diazaspiro[4.5]dec-3-en-2-one;
3-(4-chlorophenyl)-1-{2-[4-chloro-3-(trifluoromethyl)phenyl]-2-oxoethyl}-1,4-diazaspiro[4.5]dec-3-en-2-one;
3-(4-chlorophenyl)-1-{2-oxo-2-[3-(2-pyridinyl)phenyl]ethyl}-1,4-diazaspiro[4.5]dec-3-en-2-one;
3-(4-chlorophenyl)-1-{2-[4-chloro-3-(2-pyridinyl)phenyl]-2-oxoethyl}-1,4-diazaspiro[4.5]dec-3-en-2-one;
3-[4-(methyloxy)phenyl]-1-{2-oxo-2-[3-(2-pyridinyl)phenyl]ethyl}-1,4-diazaspiro[4.5]dec-3-en-2-one;
1-{2-[4-chloro-3-(2-pyridinyl)phenyl]-2-oxoethyl}-3-[4-(methyloxy)phenyl]-1,4-diazaspiro[4.5]dec-3-en-2-one;
1-[2-(4-chlorophenyl)-2-oxoethyl]-3-[4-(methyloxy)phenyl]-1,4-diazaspiro[4.5]dec-3-en-2-one;
4-{4-[2-(4-chloro-3-methylphenyl)-2-oxoethyl]-3-oxo-1,4-diazaspiro[4.5]dec-1-en-2-yl}benzonitrile;
4-(3-oxo-4-{2-oxo-2-[3-(trifluoromethyl)phenyl]ethyl}-1,4-diazaspiro[4.5]dec-1-en-2-yl)benzonitrile;
1-{2-[4-chloro-2-(methyloxy)phenyl]-2-oxoethyl}-3-(4-chlorophenyl)-1,4-d iazaspiro[4.5]dec-3-en-2-one;
1-{2-[4-ch loro-2-(methyloxy)phenyl]-2-oxoethyl}-3-[4-(methyloxy)phenyl]-1,4-diazaspiro[4.5]dec-3-en-2-one;
1-{2-[5-fluoro-2-(methyloxy)phenyl]-2-oxoethyl}-3-[4-(methyloxy)phenyl]-1,4-diazaspiro[4.5]dec-3-en-2-one;
1-[2-(3-bromo-4-chlorophenyl)-2-oxoethyl]-3-(4-chlorophenyl)-1,4-diazaspiro[4.5]dec-3-en-2-one;
2-chloro-5-{[3-(4-chlorophenyl)-2-oxo-1,4-diazaspiro[4.5]dec-3-en-1 -yl]acetyl}benzonitrile;
3-{[3-(4-chlorophenyl)-2-oxo-1,4-diazaspiro[4.5]dec-3-en-1 -yl]acetyl}benzonitrile;
3-({3-[4-(methyloxy)phenyl]-2-oxo-1,4-diazaspiro[4.5]dec-3-en- 1-yl}acetyl)benzonitrile;
1-{2-[4-chloro-3-(2-pyridinyl)phenyl]-2-oxoethyl}-3-[4-(trifluoromethyl)phenyl]-1,4-diazaspiro[4.5]dec-3-en-2-one;
3-(4-chlorophenyl)-1-{2-[2-fluoro-5-(methyloxy)phenyl]-2-oxoethyl}-1,4-diazaspiro[4.5]dec-3-en-2-one;
3-(4-chlorophenyl)-1-[2-(3,5-difluorophenyl)-2-oxoethyl]-1,4-diazaspiro[4.5]dec-3-en-2-one;
3-{[3-(4-chlorophenyl)-2-oxo-1,4-diazaspiro[4.5]dec-3-en- 1-yl]acetyl}-4-(methyloxy)benzonitrile;
4-(methyloxy)-3-({3-[4-(methyloxy)phenyl]-2-oxo-1,4-diazaspiro[4.5]dec-3-en-1 -yl}acetyl)benzonitrile;
3-(4-chlorophenyl)-1-[2-(1 -naphthalenyl)-2-oxoethyl]-1,4-diazaspiro[4.5]dec-3-en-2-one;
3-(4-chlorophenyl)-1-[2-(4-fluoro-3-methylphenyl)-2-oxoethyl]-1,4-diazaspiro[4.5]dec-3-en-2-one;
1-[2-(2-chloro-3-methylphenyl)-2-oxoethyl]-3-(4-chlorophenyl)-1,4-diazaspiro[4.5]dec-3-en-2-one;
1-{2-[4-chloro-3-(methyloxy)phenyl]-2-oxoethyl}-3-(4-chlorophenyl)-1,4-d iazaspiro[4.5]dec-3-en-2-one;
3-(4-chlorophenyl)-1-{2-[3-methyl-2-(methyloxy)phenyl]-2-oxoethyl}-1,4-diazaspiro[4.5]dec-3-en-2-one;
1-[2-(4-chloro-3-methylphenyl)-2-oxoethyl]-3-[4-(trifluoromethyl)phenyl]-1 4-diazaspiro[4.4]non-3-en-2-one;
3-(4-chlorophenyl)-1-{2-[3-fluoro-2-(methyloxy)phenyl]-2-oxoethyl}-1,4-diazaspiro[4.5]dec-3-en-2-one;
1-{2-[4-chloro-5-methyl-2-(methyloxy)phenyl]-2-oxoethyl}-3-(4-chlorophenyl)-1,4-diazaspiro[4.5]dec-3-en-2-one
4-{4-[2-(3-bromo-4-chlorophenyl)-2-oxoethyl]-3-oxo-1,4-diazaspiro[4.4]non-1-en-2-yl}benzonitrile;
2-chloro-5-{[3-(4-cyanophenyl)-2-oxo-1,4-diazaspiro[4.4]non-3-en-1-yl]acetyl}benzonitrile;
4-{4-[2-(4-chloro-3-methylphenyl)-2-oxoethyl]-3-oxo-1,4-diazaspiro[4.4]non-1-en-2-yl}benzonitrile;
4-(4-{2-[4-chloro-3-(trifluoromethyl)phenyl]-2-oxoethyl}-3-oxo-1,4-diazaspiro[4.4]non- 1-en-2-yl)benzonitrile;
4-{4-[2-(3,4-dich lorophenyl)-2-oxoethyl]-3-oxo-1,4-diazaspiro[4.4]non- 1-en-2-yl}benzonitrile; and
4-{4-[2-(3,4-dimethylphenyl)-2-oxoethyl]-3-oxo-1,4-diazaspiro[4.4]non-1-en-2-yl}benzonitrile;

and salts and solvates thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

As referred to above, individual enantiomers of compounds of formula (I) may be prepared and an indication of the preferred stereochemistry for such enantiomers has been given. In one embodiment, an optically pure enantiomer is provided. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protectinq Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic ComPounds by E. L. Eliel, S. H. Wilen, and L. N. Mander (Wiley-Interscience, 1994).

In another aspect, there is provided a process for the formation of a compound of formula (I) or a salt or solvate thereof, comprising reacting a compound of formula (II) with a base in a suitable solvent followed by reaction with a compound of formula (III)

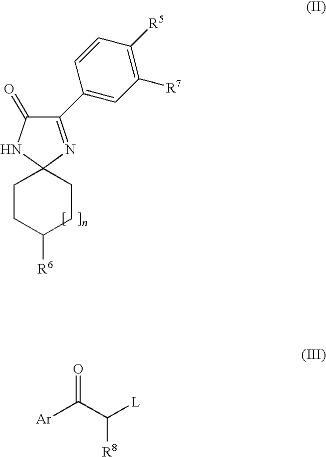

wherein Ar, n, R⁵, R⁶, R⁷ and R⁸ are as defined in Formula (I) and L is a leaving group.

In one embodiment, the base is sodium hydride.

In one embodiment, the leaving group L is halogen.

In one embodiment, the solvent is dimethylformamide.

Typical reaction routes for the preparation of a compound of formula (I) as hereinbefore defined are shown below.

Compounds of formula (I) can be prepared by reacting a compound of formula (II) with a base, for example sodium hydride, in a suitable inert solvent, for example dimethylformamide, followed by treatment with a compound of formula (III), where L is a leaving group such as halogen, as shown in Scheme 1.

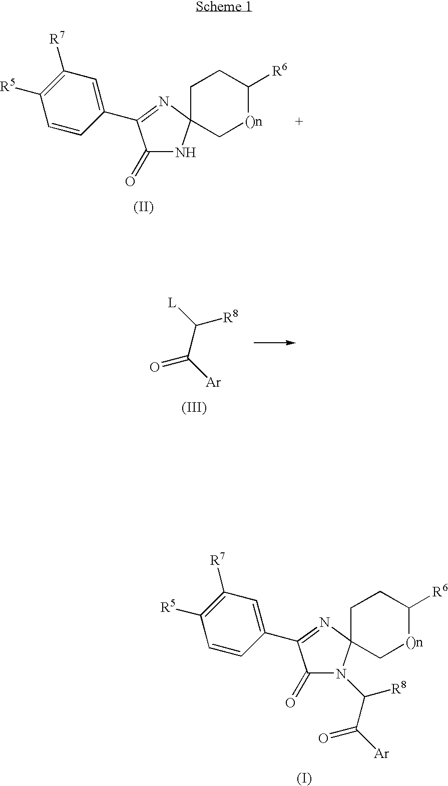

Compounds of formula (III) can be prepared by standard methods, for example as shown in Scheme 2. For example an amide of formula (V) may be reacted with a suitable organometallic reagent, for example methylmagnesium bromide or ethylmagnesium bromide, in an inert solvent, for example tetrahydrofuran, to afford the acetophenone (IV) which can be converted to a compound of formula (III), for example where L is a halogen, the acetophenone (IV) can be halogenated, for example, with bromine, optionally in the presence of aqueous hydrogen bromide, in a solvent, such as acetic acid, to give a compound of formula (III).

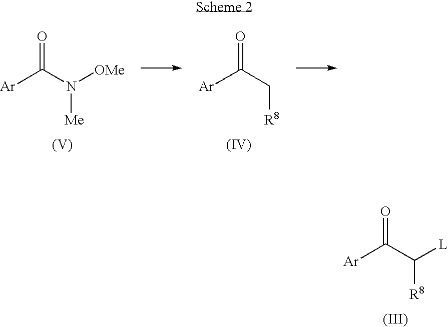

Compounds of formula (II) may be prepared by desulphurisation of compounds of formula (VI) using an oxidising agent, for example hydrogen peroxide as shown for example in Scheme 3.

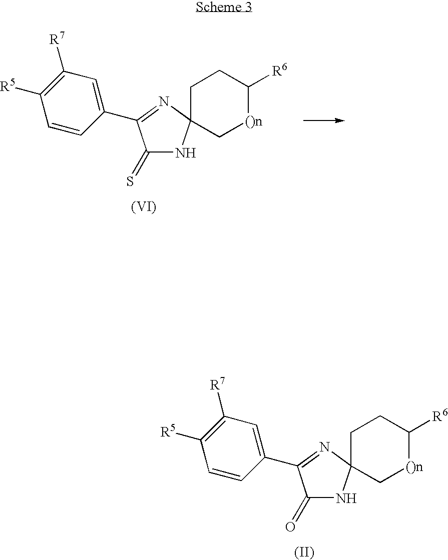

Compounds of formula (VI) can be prepared by treating a ketothioamide of formula (VII) with the appropriate ketone of formula (VIII) in the presence of a source of ammonia, for example ammonium acetate as shown in Scheme 4. Preferably this reaction is performed in a solvent, for example isopropanol, at room or elevated temperature, preferably elevated temperature, for example at reflux.

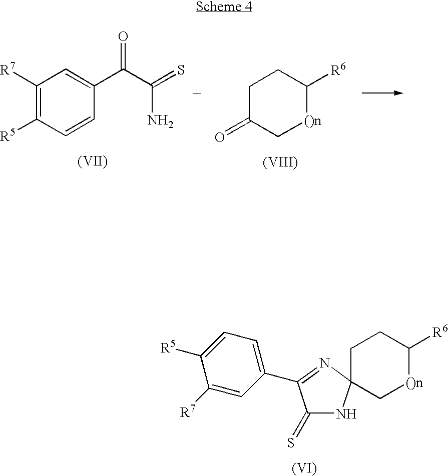

Thioamides of formula (VII) can be prepared from acylnitriles of formula (IX) by treating with, for example hydrogen sulphide in the presence of an organic base, for example triethylamine in an inert solvent, for example diethyl ether at room temperature.

Acylnitriles of formula (IX) can be prepared from the appropriate acid chloride (X) and a source of cyanide, conveniently copper (I) cyanide, at elevated temperatures, for example greater than 150° C. preferably in the absence of solvent.

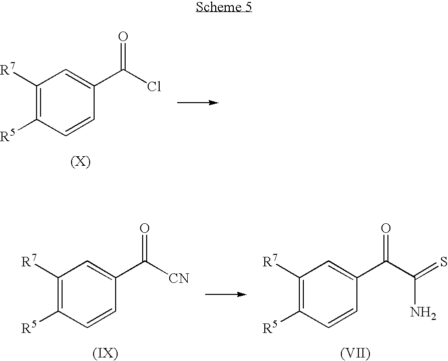

Alternatively, compounds of formula (II) can be synthesised as shown in Scheme 6.

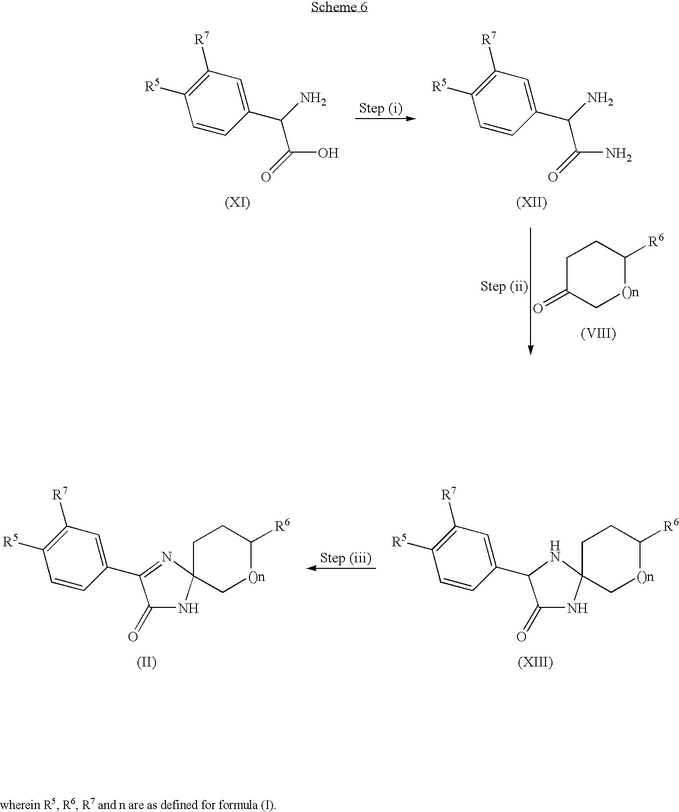

The arylglycine of formula (XI) can be converted, step (i), to the corresponding arylglycinamide of formula (XII) by standard methods, for example, by reaction of compounds of formula (XI) with thionyl chloride or acetyl chloride in methanol, followed by subsequent reaction of the intermediate methyl ester hydrochloride with aqueous ammonia.

Arylglycinamides of formula (XII) can be converted to compounds of formula (XIII), step (ii), by condensation with ketones of formula (VIII), for example, by heating in an inert solvent such as methanol, in the presence or absence of a catalyst such as H-Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Within the scheme there is scope to convert a group R¹ into another group R¹ and similarly for groups R², R³, R⁴, R⁵, R⁶, R⁷ and R⁸.

Compounds of formula (I) can be converted into further compounds of formula (I) using standard techniques.

Salts may be prepared conventionally by reaction with the appropriate acid or acid derivative.

The compounds of the present invention inhibit the GlyT1 transporter. The compounds may selectively inhibit the GlyT1 transporter over the GlyT2 transporter. Some compounds of the invention may have mixed GlyT-1/GlyT-2 activity.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assay.

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of LeadseekerTM WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. pIC₅₀ values were determined using ActivityBase.

Compounds may have activity at the the GlyT1 transporter if they have a pIC₅₀ of >5.0. The example compounds below and the individually named compounds above were found to have a pIC₅₀ at the GlyT1 transporter of greater than 5.0. Some compounds of the invention were found to have a pIC₅₀ at the GlyT1 transporter of greater than 6.0. Advantageously, compounds of the invention were found to have a pIC₅₀ at the GlyT1 transporter of greater than 7.0.

Accordingly, in one aspect of the invention, there is provided a compound of formula (I) as hereinbefore described or a salt or solvate thereof, for use as a medicament. In a further aspect of the invention, there is provided a compound of formula (I) as hereinbefore described or a salt or solvate thereof, for use in the treatment of a disorder mediated by GlyT1.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

In another aspect of the invention, there is provided use of a compound of formula (I) as hereinbefore defined or a salt or solvate thereof in the preparation of a medicament for the treatment of a disorder mediated by GlyT1.

In one embodiment, the disorder mediated by GlyT1 to be treated by the use or method as hereinbefore described is a psychosis, including schizophrenia, dementia and attention deficit disorders, particularly schizophrenia.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient.

In another aspect of the invention, there is provided a process of preparing a pharmaceutical composition, which process comprises mixing a compound of formula (I) or a salt or solvate thereof, together with a pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other pharmaceutically active ingredients may also be incorporated in the pharmaceutical compositions of the invention.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

The compounds of formula (I) may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) may also be of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic- Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) may also be of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of formula (I) may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of formula (I) may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The invention also provides a compound of formula (I) as hereinbefore described or a salt or solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a salt or solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

The invention also provides a method of treating schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a salt or solvate thereof.

The invention also provides a method of treating psychotic disorders, substance abuse, anxiety, and gastric motility disorders which comprises administering to a mammal in need thereof an effective amount of a compound of formula (I) as hereinbefore described or a salt or solvate thereof.

The compounds of formula (I) may also be of use as anticonvulsants. The compounds of formula (I) are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a salt or solvate thereof. Treatment of epilepsy may be carried out by the administration of a non-toxic anticonvulsant effective amount of a compound of the formula (I) or a salt, or a composition as hereinbefore defined.

The compounds of formula (I) may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

Other disorders include benign forgetfulness, childhood learning disorders and closed head injury, Parkinson's disease, dyskinetic disorders, cognitive impairment, emesis, movement disorders, amnesia, circadian rhythm disorders, aggression and vertigo.

In another aspect of the invention, there is provided use of a compound of formula (I) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

The invention also provides the use of a compound of formula (I) as hereinbefore described or a salt or solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a salt or solvate thereof in the manufacture of a medicament for the treatment of psychotic disorders, substance abuse, autism, and gastric motility disorders.

As used herein, the term “effective amount” means that amount of a drug or pharmaceutical agent that will elicit the biological or medical response of a tissue, system, animal or human that is being sought, for instance, by a researcher or clinician.

It will be appreciated that the compounds of the combination or composition may be administered simultaneously (either in the same or different pharmaceutical formulations), separately or sequentially.

The compounds of formula (I) and their salts and solvates thereof are also suitable for combination with other typical and atypical antipsychotics to provide improved treatment of psychotic disorders. Particular advantages associated with the combinations, uses and methods of treatment of compounds of formula (I) and their salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain neuroleptic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt or solvate thereof and at least one neuroleptic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, it is provided that the compounds of formula (I) or a salt or solvate thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of compounds of formula (I) or a salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt or solvate thereof to a patient receiving therapeutic administration of at least one neuroleptic agent. In a further aspect, the invention provides the use of compounds of formula (I) or a salt or solvate thereof in the manufacture of a medicament for adjunctive therapeutic administration for the treatment of a psychotic disorder in a patient receiving therapeutic administration of at least one neuroleptic agent. The invention further provides compounds of formula (I) or a salt or solvate thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of compounds of formula (I) or a salt or solvate thereof. In a further aspect, the invention provides the use of at least one neuroleptic agent in the manufacture of a medicament for adjunctive therapeutic administration for the treatment of a psychotic disorder in a patient receiving therapeutic administration of compounds of formula (I) or a salt or solvate thereof. The invention further provides at least one neuroleptic agent for adjunctive therapeutic administration for the treatment of a psychotic disorder in a patient receiving therapeutic administration of compounds of formula (I) or a salt or solvate thereof.

In a further aspect, the invention provides a method of treatment of a psychotic disorder by simultaneous therapeutic administration of compounds of formula (I) or a salt or solvate thereof in combination with at least one neuroleptic agent. The invention further provides the use of a combination of compounds of formula (I) or a salt or solvate thereof and at least one neuroleptic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one neuroleptic agent in the treatment of a psychotic disorder. The invention further provides compounds of formula (I) or a salt thereof for use for simultaneous therapeutic administration with at least one neuroleptic agent in the treatment of a psychotic disorder. The invention further provides the use of at least one neuroleptic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt or solvate thereof and at least one mood stabilising or antimanic agent, a pharmaceutical composition comprising compounds of formula (I) or a salt or solvate thereof and at least one mood stabilising or antimanic agent, the use of a pharmaceutical composition comprising compounds of formula (I) or a salt or solvate thereof and at least one mood stabilising or antimanic agent in the manufacture of a medicament for the treatment of a psychotic disorder, and a pharmaceutical composition comprising compounds of formula (I) or a salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a salt or solvate thereof and one or more further dosage forms each comprising a neuroleptic agent for simultaneous therapeutic administration.

Examples of neuroleptic/antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected neuroleptic drugs are as follows: clozapine (available under the tradename CLOZARIL®, from Mylan, Zenith Goldline,UDL, Novartis); olanzapine (available under the tradename ZYPREX®, from Lilly); ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK)); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Kline Beckman); perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment oral administration is provided.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution is preferably isotonic with the blood of the intended recipient. Although such solutions are preferably administered intravenously, they may also be administered by subcutaneous or intramuscular injection.

Formulations suitable for rectal administration are preferably provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

The invention is further illustrated by the following non-limiting examples.

Starting materials were obtained from commercial suppliers and used without further purification unless otherwise stated. Flash chromatography was carried out using pre-packed Isolute Flash™ or Biotage™ silica-gel columns as the stationary phase and analytical grade solvents as the eluent.

NMR spectra were obtained at 298K, at the frequency stated using either a BrukerTM DPX400 or an Oxford Instruments™ 250 MHz machine and run as a dilute solution of CDCl₃ unless otherwise stated. All NMR spectra were referenced to tetramethylsilane (TMS δ_{H} 0, δ_{c} 0). All coupling constants are reported in hertz (Hz), and multiplicities are labelled s (singlet), bs, (broad singlet), d (doublet), t (triplet), q (quartet), dd (doublet of doublets), dt (doublet of triplets) and m (multiplet).

Total ion current traces were obtained for electrospray positive and negative ionisation (ES+/ES−) and atmospheric pressure chemical positive and negative ionisation (AP+/AP−).

All quoted retention times are as measured using LC/MS (Liquid Chromatography/Mass Spectrometry). Where appropriate, these retention times were used as a guide for purification using mass-directed auto-preparation (MDAP), which refers to purification by HPLC, wherein fraction collection is triggered by detection of the programmed mass ion for the compound of interest.

Unless otherwise stated, all compounds with chiral centre(s) are racemic.

In the procedures that follow, reference to an Intermediate or Example by number is typically provided. This is provided merely for assistance to the skilled chemist to identify the starting material used. The starting material may not necessarily have been prepared from the batch referred to. All reactions were either carried out under argon or may be carried out under argon, unless otherwise stated.

Abbreviations:

THF tetrahydrofuran

DCM dichloromethane

DMF dimethylformamide

MeOH methanol

DMSO dimethylsulfoxide

Analytical LC/MS chromatography conditions:

| | |
|---|---|
| Column: | Waters Atlantis 50 mm × 4.6 mm, 3 μm |
| | particle size |
| Mobile phase: | A: 0.05% Formic acid + Water |
| | B: Acetonitrile + 0.05% Formic acid |
| Gradient: | 5-min runtime: 3% B to 97% B over 4 min |
| Flow rate: | 3 ml/min |
| UV wavelength range: | 220-330 nm |
| Temperature: | 30° C. |

Mass Directed Auto-Purification (MDAP) System chromatography conditions:

| | |
|---|---|
| Column: | Waters Atlantis 19 mm × 100 mm or 30 mm × |
| | 100 mm, 5 μm particle size |
| Mobile phase: | A: 0.1% Formic acid + Water |
| | B: Acetonitrile + 0.1% Formic acid |
| Gradient: | 13.5 min runtime with 10 min gradient |
| | dependant on analytical retention time |
| Flow rate: | 20 or 40 ml/min |

### General:

Where reactions are described as having been carried out in a similar manner to earlier, more completely described reactions, the general reaction conditions used were essentially the same. Work up conditions used were of the types standard in the art, but may have been adapted from one reaction to another.

### DESCRIPTIONS AND EXAMPLES

Description 1: Methyl amino(4-chlorophenvl)acetate hydrochloride

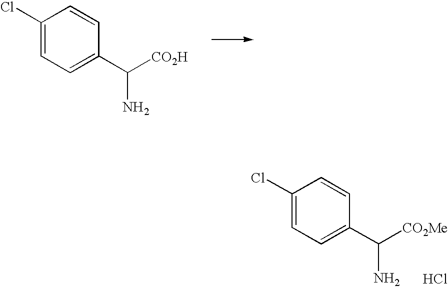

To ice-chilled methanol (300 ml) under argon was carefully added dropwise thionyl chloride (15.44 ml; 0.217mol) over 45 min. 4-Chlorophenylglycine (26.26 g; 0.141 mol) was added, ice cooling removed and the reaction mixture warmed to 40° C.; the reaction was stirred at 40° C. for 48 h. After cooling to room temperature, the reaction was evaporated under reduced pressure. Re-evaporation from methanol afforded a white solid which was triturated with diethyl ether (ca. 700 ml) and then stored at ca. 4° C. for 64 h, filtered, washed with diethyl ether and dried in vacuo to afford the title product as the hydrochloride salt (33.40 g; 100%). ¹H NMR (d₆-DMSO) δ: 3.72 (3H, s), 5.36 (1H, s), 7.53-7.58 (4H, m), 9.07 (3H, s). Mass Spectrum (Electrospray LC/MS): Found 200 (MH⁺). C₉H₁₀³⁵CINO₂ requires 199. Ret. time 1.32 min.
Description 2: 2-Amino-2-(4-chlorophenyl)acetamide

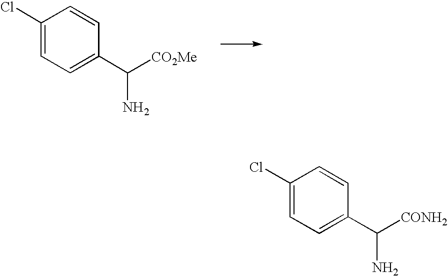

Methyl amino(4-chlorophenyl)acetate D1 as the hydrochloride salt (33.40 g; 0.14 mol) was dissolved in 0.88 ammonia (500 ml; ca. 7.4 mol) and stirred at room temperature for 64 h. The reaction mixture was extracted with DCM (300 ml×6), the extracts dried (Na₂SO₄) and evaporated under reduced pressure to afford a white solid, which was dried under reduced pressure to afford the title product (22.45 g; 86%). ¹H NMR (CDCl₃) δ: 1.82 (2H, br s), 4.53 (1H, s), 5.49 (1H, br s), 6.92 (1H, br s), 7.32-7.39 (4H, m).
Description 3: 3-(4-Chlorophenyl)-1,4-diazaspiro[4.5]decan-2-one

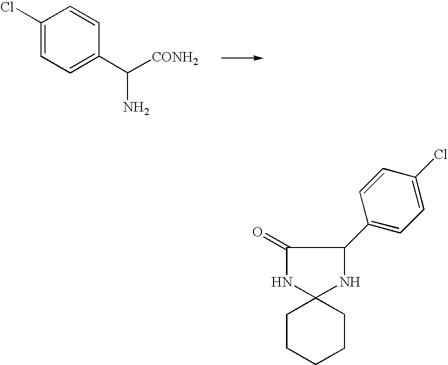

To 2-amino-2-(4-chlorophenyl)acetamide D2 (10.00 g; 54.3 mmol) in methanol (500 ml) was added cyclohexanone (5.62 ml; 54.3 mmol) and H-Y zeolites (10.00 g) and the mixture stirred under reflux for 24 h. The reaction was allowed to cool to room temperature and after 4 days was filtered and the solid washed well with methanol. The filtrate was evaporated to afford the title product (12.91 g; 90%) as a white solid. ¹H NMR (CDCl₃) δ: 1.44-1.57 (4H, m), 1.66-1.76 (6H, m), 2.21 (1H, s), 4.69 (1H, s), 6.80 (1H, s), 7.32-7.35 (2H, m), 7.45 -7.49 (2H, m).
Description 4: 3-(4-Chlorophenyl)-1,4-diazaspiro[4.5]dec-3-en-2-one

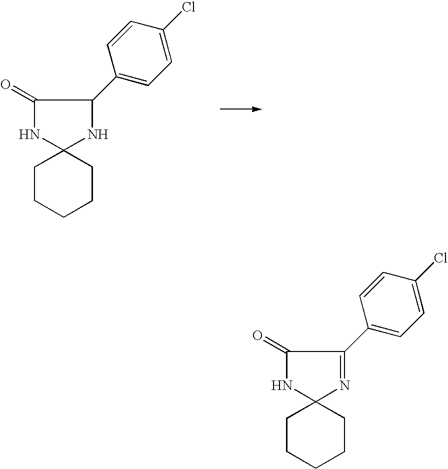

N-Bromosuccinimide (8.69 g; 48.81 mmol) was added in one portion to a stirred solution of 3-(4-chlorophenyl)-1,4-diazaspiro[4.5]decan-2-one D3 (12.91 g; 48.81 mmol) in DCM (400 ml) and the mixture stirred overnight at room temperature. Saturated aqueous sodium bicarbonate (500 ml) was added and the mixture stirred for 0.5 h. The layers were separated and the aqueous extracted with DCM (300 ml). The combined organics were dried (Na₂SO₄) and the solvent removed under reduced pressure at 45° C. The residual solid was partitioned between DCM (500 ml) and saturated aqueous sodium bicarbonate (500 ml) and stirred overnight at room temperature. The aqueous layer was extracted with DCM (300 ml) and the organic layers combined, dried (Na₂SO₄) and the solvent removed under reduced pressure to afford the title product (10.25 g; 80%) as a pale yellow solid. ¹H NMR (CDCl₃) δ: 1.51-1.70 (6H, m), 1.91-1.99 (4H, m), 7.42-7.49 (2H, m), 8.36-8.39 (2H, m), 8.88 (1H, s).
Description 5: 2-Amino-2-[4-(methyloxy)phenyl]acetamide

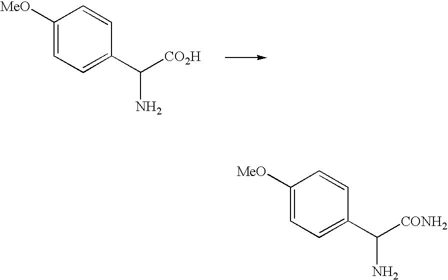

To an ice-cold suspension of 4-methoxyphenylglycine (3.77 g; 0.021 mol) in methanol was added thionyl chloride dropwise over 30 min. After complete addition, the reaction mixture was heated at reflux for 3 h, cooled and evaporated. The resulting solid was dissolved in 0.88 ammonia (100 ml) and stirred at room temperature overnight. The reaction was extracted twice with DCM and the organic phases separated with a Phase-Separation cartridge and evaporated under reduced pressure to afford the title product (0.45 g; 12%) as a white solid. ¹H NMR (CDCl₃) δ: 1.77 (2H, br s), 3.80 (3H, s), 4.50 (1H, s), 5.52 (1H, s), 6.83 (1H, s), 6.87-6.91 (2H, m), 7.33-7.36 (2H, m).
Description 6: 3-[4-(Methyloxy)phenyl]-1,4-diazaspiro[4.5]decan-2-one

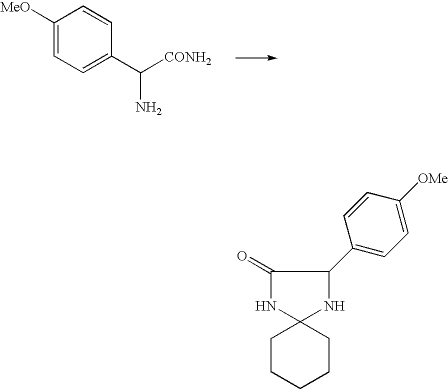

The title compound (0.420 g; 65%) was obtained from 2-amino-2-[4-(methyloxy)phenyl]acetamide D5 (0.450 g; 2.5 mmol), cyclohexanone (0.245 g; 2.5 mmol) and H-Y zeolites (1 g) in methanol (20 ml) using a similar method to that described in D3. ¹H NMR (CDCl₃) δ: 1.44-1.57 (4H, m), 1.71-1.73 (6H, m), 2.11 (1H, brs), 3.80 (3H, s), 4.64 (1H, s), 6.55 (1H, br s), 6.89-6.92 (2H, m), 7.36-7.40 (2H, m).
Description 7: 3-[4-(Methyloxy)phenyl]-1,4-diazaspiro[4.5]dec-3-en-2-one

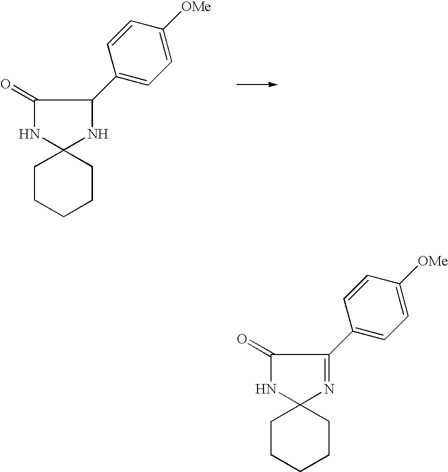

The title product (406 mg; 100%) was obtained from 3-[4-(methyloxy)phenyl]-1,4-diazaspiro[4.5]decan-2-one D6 (400 mg; 1.54 mmol) and N-bromosuccinimide (275 mg; 1.55 mmol) in DCM (20 ml) using a similar method to that described in D4. ¹H NMR (CDCl₃) δ: 1.40-1.75 (6H, m), 1.85-2.00 (4H, m), 3.87 (3H, s), 6.94-6.98 (2H, m), 8.18 (1H, brs), 8.37-8.40 (2H, m).
Description 8: Methyl amino[4-(trifluoromethyl)phenyl]acetate hydrochloride

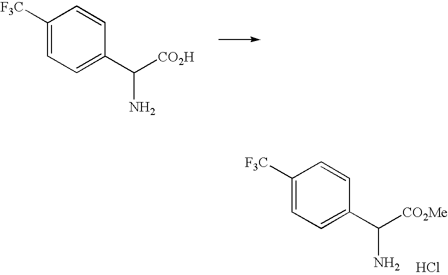

Thionyl chloride (5 ml; 68.9 mmol) was added dropwise under argon to methanol (100 ml) chilled in an ice-bath over 45 min. 4-Trifluoromethylphenylglycine (10 g; 45.6 mmol) was added and the mixture heated at 40° C. for 40 h. After cooling to room temperature, the reaction was evaporated under reduced pressure. The resulting solid was dissolved in methanol (200 ml) and evaporated. Diethylether (250 ml) was added and the product filtered and dried to afford the title compound as the hydrochloride salt (12 g; 98%). ¹H NMR (d₆-DMSO) δ: 3.74 (3H, s), 5.52 (1H, s), 7.74 (2H, d, J=8 Hz), 7.89 (2H, d, J=8 Hz), 9.00 (3H, br s). Mass Spectrum (Electrospray LC/MS) Found 234 (MH⁺). C₁₀H₁₀F₃NO₂ requires 233. Ret. time 1.55 min.
Description 9: 2-Amino-2-[4-(trifluoromethyl)phenyl]acetamide

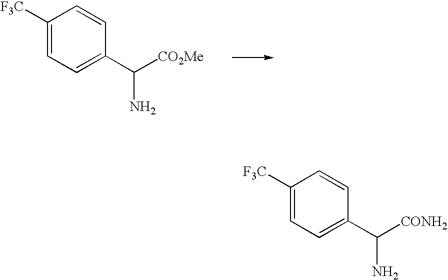

Methyl amino[4-(trifluoromethyl)phenyl]acetate D8 as the hydrochloride salt (12 g; 44.5 mmol) was dissolved in 0.88 ammonia (220 ml; ca. 3.3 mol). After stirring overnight the reaction mixture was extracted with DCM (150 ml×5) and the extracts dried with Na₂SO₄ and the solvent evaporated to afford the title compound (8.92 g; 92%) ¹H NMR (CDCl₃) δ: 1.87 (2H, br s), 4.62 (1H, s), 5.48 (1H, br s), 7.00 (1H, br s) 7.57 (2H, d, J=8 Hz), 7.63 (2H, d, J=8 Hz). Mass Spectrum (Electrospray LC/MS) Found 219 (MH⁺). C₉H₉F₃N₂O requires 218. Ret. time 1.13 min.
Description 10: 3-[4-(Trifluoromethyl)phenyl]-1,4-diazaspiro[4.5]decan-2-one

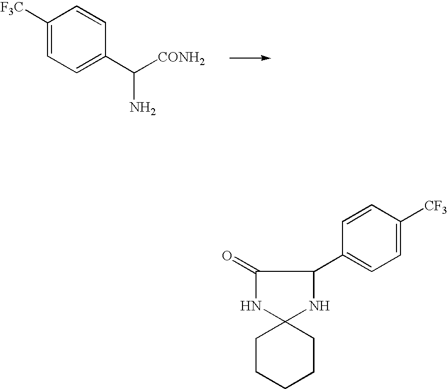

To 2-amino-2-[4-(trifluoromethyl)phenyl]acetamide D9 (8.92 g; 40.9 mmol), in methanol (350 ml) was added cyclohexanone (4.24 ml; 40.9 mmol) and H-Y zeolites (8.92 g) and the mixture refluxed overnight. After cooling to room temperature and chilling in an ice-bath the reaction mixture was filtered. The solid was washed with methanol and the filtrate evaporated under reduced pressure to afford the title compound (10.59 g; 86%) ¹H NMR (CDCl₃) δ: 1.35-1.60 (4H, m), 1.62-1.80 (6H, m), 2.31 (1H, br s), 4.79 (1H, br s) 6.41 (1H, br s), 7.62 (2H, d, J=8 Hz), 7.70 (2H, d, J=8 Hz). Mass Spectrum (Electrospray LC/MS) Found 299 (MH⁺). C₁₅H₁₇F₃N₂O requires 298. Ret. time 2.57 min.
Description 11: 3-[4-(Trifluoromethyl)phenyl]-1,4-diazaspiro[4.5]dec-3-en-2-one

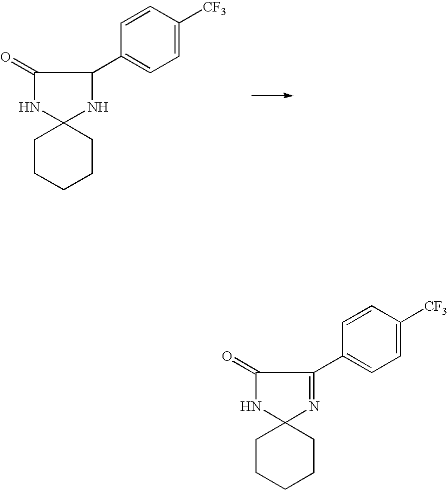

N-Bromosuccinimide (6.32 g; 35.5 mmol) was added to 3-[4-(trifluoromethyl)phenyl]-1,4-diazaspiro[4.5]decan-2-one D10 (10.59 g; 35.5 mmol) in DCM (200 ml) and the reaction stirred overnight at room temperature under argon. Saturated aqueous sodium bicarbonate (150 ml) was added and the mixture stirred, the organic layer was then separated and the aqueous extracted with DCM. The combined DCM extracts were dried with Na₂SO₄, filtered and evaporated under reduced pressure to afford the title product (5 g). Additional washing of the filtered Na₂SO₄ with methanol-DCM afforded further title product, giving 10.69 g in total. Mass Spectrum (Electrospray LC/MS) Found 297 (MH⁺). C₁₅H₁₅F₃N₂O requires 296. Ret. time 3.14 min.
Description 12: Methyl amino{4-[(trifluoromethyl)oxy]phenyl}acetate hydrochloride

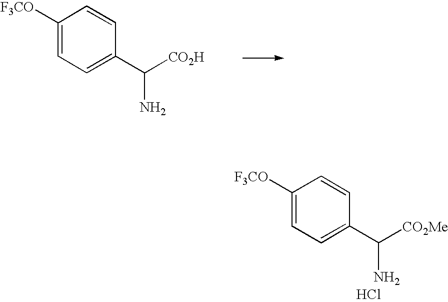

To ice-chilled methanol (30 ml) under argon was carefully added dropwise thionyl chloride (15.44 ml; 0.422 mol) over 30 min. Amino{4-[(trifluoromethyl)oxy]phenyl}acetic acid (5.0 g; 21.280 mmol) was added, ice cooling removed and the reaction mixture was stirred at room temperature for 16 h. The reaction was then evaporated under reduced pressure. Trituration with diethyl ether, followed by filtration provided the title product as the hydrochloride salt, (5.75 g; 95%). ¹H NMR (d₆-DMSO) δ: 3.74 (3H, s), 5.41 (1H, s), 7.51 (2H, d), 7.66 (2H, d), 9.10 (3H, s). Mass Spectrum (Electrospray LC/MS): Found 250 (MH⁺). C₁₀H₁₀F₃NO₃ requires 249. Ret. time 1.52 min.
Description 13: 2-Amino-2-{4-[(trifluoromethyl)oxy]phenyl}acetamide

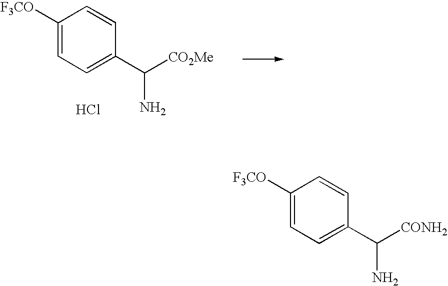

Methyl amino{4-[(trifluoromethyl)oxy]phenyl}acetate D12 as the hydrochloride salt (5.75 g; 20.14 mMol) was dissolved in 0.88 ammonia (75 ml; ca. 1.1 mol) and stirred at room temperature under argon for 16 h. The reaction mixture was extracted with DCM, the extracts dried (MgSO₄) and evaporated under reduced pressure to a white solid, which was dried under reduced pressure to afford the title product (3.70 g; 79%). ¹H NMR (d₆-DMSO) δ: 2.22 (2H, br s), 4.32 (1H, s), 7.08 (1H, br s), 7.30 (2H, d), 7.50 (3H, d). Mass Spectrum (Electrospray LC/MS): Found 235 (MH⁺). C₉H₉F₃N₂O₂ requires 234. Ret. time 1.20 min.
Description 14: 3-{4-[(Trifluoromethyl)oxy]phenyl}-1,4-diazaspiro[4.5]decan-2-one

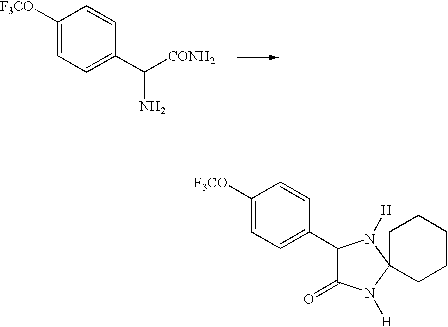

To 2-amino-2-{4-[(trifluoromethyl)oxy]phenyl}acetamide D13 (3.70 g; 15.81 mmol) in methanol (200 ml) was added cyclohexanone (1.549 ml; 15.81 mmol) and H-Y zeolites (6.00 g) and the mixture stirred under reflux for 24 h under argon. The reaction was allowed to cool to room temperature and was filtered and the solid washed well with methanol. The filtrate was evaporated to afford the title product (3.88 g; 50%) as a white solid, after trituration with hexane. ¹H NMR (d₆-DMSO) δ: 1.22-1.45 (2H, m), 1.50-1.70 (8H, m), 3.53 (1H, d), 4.64 (1H, d), 7.32 (2H, d), 7.60 (2H, d), 8.68 (1H,s). Mass Spectrum (Electrospray LC/MS): Found 315 (MH⁺). C₁₅H₁₇F₃N₂O₂ requires 314. Ret. time 2.57 min.
Description 15: 3-{4-[(Trifluoromethyl)oxy]phenyl}-1,4-diazaspiro[4.5]dec-3-en-2-one

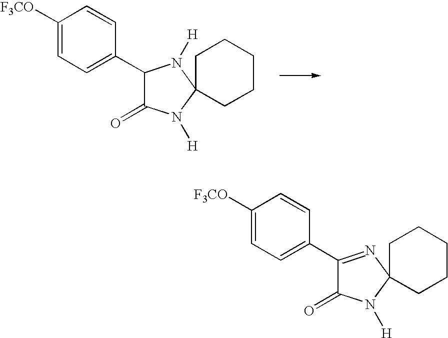

3-{4-[(Trifluoromethyl)oxy]phenyl}-1,4-diazaspiro[4.5]decan-2-one D14 (3.880 g; 12.36 mmol) was dissolved in DCM (80 ml) and stirred at room temperature for 16 hours under an atmosphere of argon with N-bromosuccinimide (2.216 g; 12.36 mmol). A solution of saturated sodium hydrogen carbonate (100 ml) was then added and stirring continued for 1 hour at room temperature. The organic layer was separated, dried (MgSO₄) and evaporated at reduced pressure to yield the title compound as a yellow solid after trituration with hexane (3.25 g; 84%). ¹H NMR (d₆-DMSO) δ: 1.40-1.85 (10H, m), 7.50 (2H, d), 8.47 (2H, d),10.30 (1H,s). Mass Spectrum (Electrospray LC/MS): Found 313 (MH⁺). C₁₅H₁₅F₃N₂O₂ requires 312. Ret. time 3.23 min.
Description 16: 2-Amino-2-(4-bromophenyl)acetamide

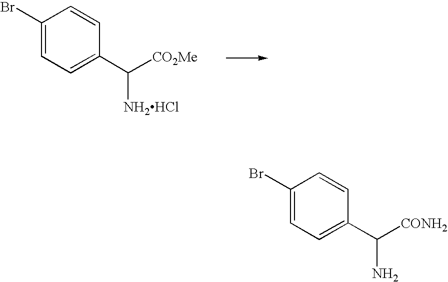

Methyl amino(4-bromophenyl)acetate hydrochloride (commercially available from Bionet Research) (5.0 g; 17.822 mmol) was elaborated to the title compound (2.69 g; 66%) using concentrated ammonia solution (75 ml) using a similar procedure to that described in D13. ¹H NMR (d₆-DMSO) δ: 2.20 (2H, brs), 4.38 (1H, s), 7.08 (1H, brs), 7.36 (2H, d), 7.50 (3H, d).

Alternative Procedure

Methyl amino(4-bromophenyl)acetate hydrochloride (commercially available from Bionet Research) (9.6 g) was elaborated to the title compound (6.4 g; 81%) using 0.880 ammonia solution (300 ml) using a similar procedure to that described in D13. ¹H NMR (d₆-DMSO) δ: 2.20 (2H, brs), 4.38 (1H, s), 7.08 (1H, brs), 7.36 (2H, d), 7.50 (3H, d).

Description 17: 3-(4-Bromophenyl)-1,4-diazaspiro[4.4]nonan-2-one

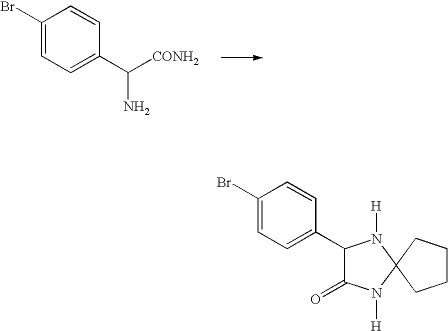

The title compound was prepared from 2-amino-2-(4-bromophenyl)acetamide D16 (2.29 g; 10 mmol), cyclopentanone (0.9 ml; 10 mmol) and H-Y zeolites (3 g) in ethanol (200 ml) using a similar procedure to that described in D10, except that further cyclopentanone (0.9 ml) and H-Y zeolites (3 g) were added after 20 hours of reflux and the heating continued for a further 24 hours. After work-up the title compound (1.91 g; 65%) was obtained as a colourless solid. Mass Spectrum (Electrospray LC/MS) Found 295 (MH⁺). C₁₃H₁₅⁷⁹BrN₂O requires 294. Ret. time 1.83 min.
Description 18: 3-(4-Bromophenyl)-1,4-diazaspiro[4.4]non-3-en-2-one

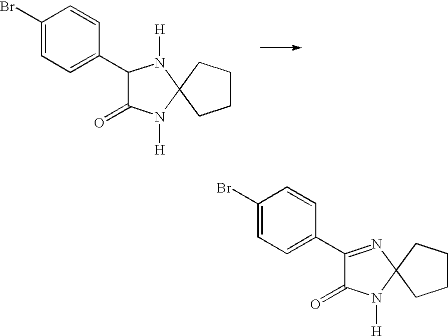

The title compound (1.80 g; 94%) was prepared from 3-(4-bromophenyl) -1,4-diazaspiro[4.4]nonan-2-one D17 (1.91 g; 6.48 mmol) and N-bromosuccinimide (1.153 g; 6.48 mmol) in DCM (150 ml) using a similar procedure to that described in D11, except that the initial reaction mixture was stirred for 66 hours instead of 16 hours and the amount of saturated sodium hydrogen carbonate used was 300 ml and the mixture was stirred for a further 2 hours following the addition of the sodium hydrogen carbonate. Mass Spectrum (Electrospray LC/MS) Found 293 (MH⁺). C₁₃H₁₃⁷⁹BrN₂O requires 292. Ret. time 2.73 min.
Description 19: 4-(3-Oxo-1,4-diazaspiro[4.4]non-1-en-2-yl)benzonitrile

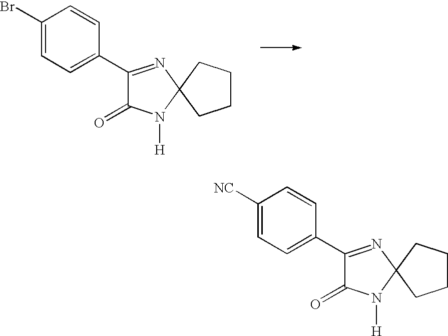

Copper (I) cyanide (0.92 g; 10.24 mmol) was added in one portion to a rapidly stirred mixture of 3-(4-bromophenyl)-1,4-diazaspiro[4.4]non-3-en-2-one D18 (1.5 g; 5.12 mmol) in NMP (25 ml) under an argon atmosphere and was heated at vigorous reflux for 3 hours. On cooling water (0.5 L) and ethyl acetate (300 ml) were added and the mixture filtered through kieselguhr. The filtrate layers were separated and the aqueous layer extracted with ethyl acetate (300 ml). Combined organics were dried (Na₂SO₄) and evaporated under reduced pressure. Chromatography on silica gel eluting with 0-100% ethyl acetate in pentane gradient gave the title compound as a pale orange solid (490 mg; 40%). Mass Spectrum (Electrospray LC/MS) Found 240 (MH+). C₁₄H₁₃N₃O. requires 239. Ret. time 2.42 min.

Description 20: 3-(4-Bromophenyl)-1,4-diazaspiro[4.5]decan-2-one

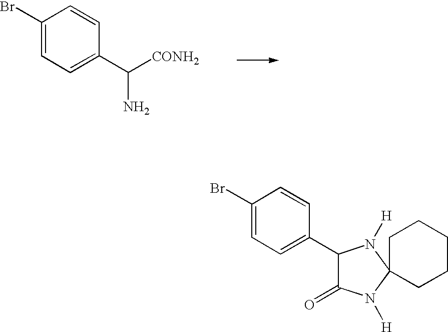

The title compound (2.22 g; 61%) was prepared from 2-amino-2-(4-bromophenyl)acetamide D16 (2.69 g; 11.75 mmol), cyclohexanone (1.22 ml; 11.75 mmol; 1 eq) and H-Y zeolites (2.69 g) in methanol (100 ml) using a similar procedure to that described in D10. ¹H NMR (d₆-DMSO) δ: 1.22-1.43 (2H, m), 1.48-1.70 (8H, m), 3.50 (1H, d), 4.58 (1H, d), 7.43 (2H, d), 7.51 (2H, d), 8.62 (1H,s).
Description 21: 3-(4-Bromophenyl)-1,4-diazaspiro[4.5]dec-3-en-2-one

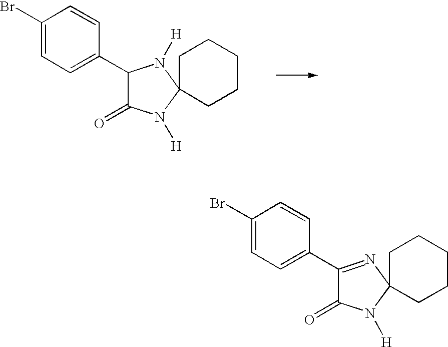

The title compound was prepared from 3-(4-bromophenyl)-1,4-diazaspiro[4.5]decan-2-one D20 (4.96 g) and N-bromosuccinimide (2.88 g; 1 eq) in DCM (100 ml) using a similar procedure to that described in D11. Yield 1.69 g. ¹H NMR (d₆-DMSO) δ: 1.42-1.88 (10H, m), 7.70 (2H, d), 8.28 (2H, d), 10.30 (1H, br s).
Description 22: 4-(3-Oxo-1,4-diazaspiro[4.5]dec-1-en-2-yl)benzonitrile

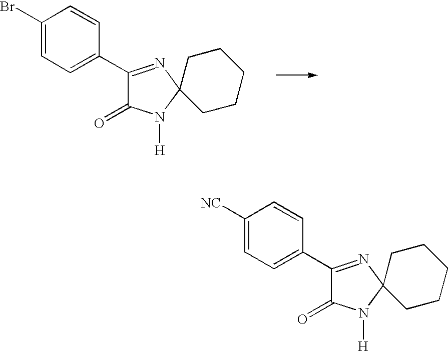

The title compound (420 mg) was prepared from 3-(4-bromophenyl)-1,4-diazaspiro[4.5]dec-3-en-2-one D21 (1.0 g; 3.26 mmol) and copper (I) cyanide (587 mg; 2 eq) in NMP (20 ml) using a similar procedure to that described in D36, except that the product was crystallised from diethyl ether/hexane to give a white solid (420 mg). From the mother liquors, an additional quantity of the title compound (0.321 mg) was obtained. Mass Spectrum (Electrospray LC/MS) Found 254 (MH⁺). C₁₅H₁₅N₃O requires 253. Ret. time 2.64 min. A further quantity of the title compound (0.406 g; 29%) was isolated from the top of the chromatography column.

### Example 1

1-[2-(4-Chloro-3-methylphenyl)-2-oxoethyl]-3-(4-chlorophenyl)-1,4-diazaspiro[4.5]dec-3-en-2-one

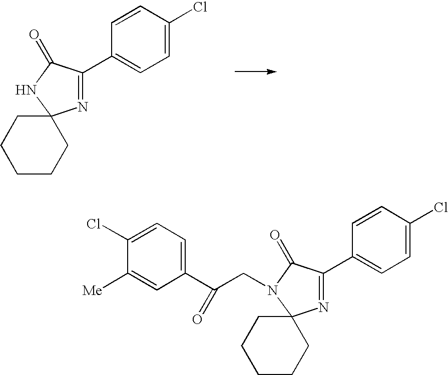

To an ice-cooled stirred solution of 3-(4-chlorophenyl)-1,4-diazaspiro[4.5]dec-3-en-2-one D4 (0.200 g; 0.76 mmol) in DMF (4 ml) under argon was added 60% sodium hydride dispersion in mineral oil (0.038 g; 0.95 mmol) in three portions over 5 minutes. After a further 20 minutes a solution of 2-bromo-1-(4-chloro-3-methylphenyl)ethanone (0.240 g; 0.98 mmol) in DMF (1 ml) was added dropwise over 2 minutes. After a further 2 hours water (100 ml) was added and the precipitate filtered, washed with water and taken up in DCM (25 ml), dried (Na₂SO₄) and the solvent removed under reduced pressure. The residue was purified by chromatography on silica eluting with 0-20% ethyl acetate in pentane gradient to afford the title compound (0.080 g; 24%). ¹H NMR (CDCl₃) δ: 1.20-1.30 (1H, m), 1.45-1.49 (2H, d), 1.70-1.78 (4H, m), 1.85-1.90 (1H, d), 1.95-2.10 (2H, m), 2.45 (3H, s), 4.80 (2H, s), 7.40-7.50 (3H, m), 7.76-7.80 (1H, m), 7.88 (1H, m), 8.44-8.48 (2H, m). Mass Spectrum (Electrospray LC/MS) Found 429 (MH⁺). C₂₃H₂₂³⁵Cl₂N₂O₂ requires 428. Ret. time 4.07 min.

### Example 2

3-(4-Chlorophenyl)-1-{2-oxo-2-[3-(trifluoromethyl)phenyl]ethyl}-1,4-diazaspiro[4.5]dec-3-en-2-one

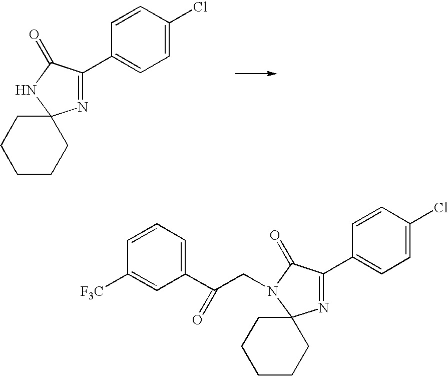

To a stirred, room temperature solution of 3-(4-chlorophenyl)-1,4-diazaspiro[4.5]dec-3-en-2-one D4 (0.129 g; 0.49 mmol) in DMF (3 ml) under argon was added 60% sodium hydride dispersion in mineral oil (0.024 g; 0.58 mmol) in one portion. After a further 5 minutes 2-bromo-1-[3-(trifluoromethyl)phenyl]ethanone (0.157 g; 0.58 mmol) was added in one portion and the resultant solution allowed to stand for 70 hours. Brine (100 ml) was added and the mixture extracted with ethyl acetate (2×50 ml). Combined organics were dried (Na₂SO₄) and the solvent removed under reduced pressure. The residue was purified by MDAP to afford the title compound as a pale orange solid (0.036 g; 16%). ¹H NMR (CDCl₃) δ: 1.20-1.33 (1H, m), 1.46-1.51 (2H, d), 1.70-1.80 (4H, m), 1.87-1.91 (1H, d), 1.95-2.10 (2H, m), 4.86 (2H, s), 7.42-7.49 (2H, m), 7.65-7.70 (1H, t, J =7.6Hz), 7.88-7.91 (1H, d, J=8 Hz), 8.20-8.23 (1H, d, J=8 Hz), 8.27 (1H, s), 8.44-8.48 (2H, m). Mass Spectrum (Electrospray LC/MS) Found 449 (MH⁺). C₂₃H₂₀³⁵ClF₃N₂O₂ requires 448. Ret. time 3.96 min.

### Example 3

1-[2-(3,4-Difluorophenyl)-2-oxoethyl]-3-[4-(trifluoromethyl)phenyl]-1,4-diazaspiro[4.5]dec-3-en-2-one

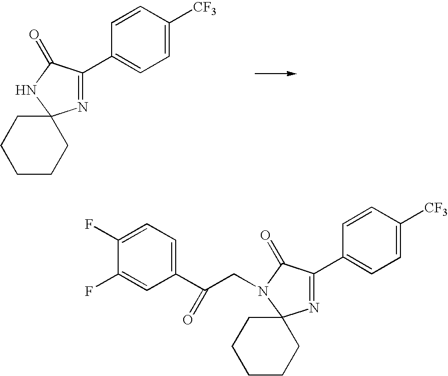

To a stirred, room temperature solution of 3-(4-trifluoromethylphenyl)-1,4-diazaspiro[4.5]dec-3-en-2-one D11 (0.118 g; 0.40 mmol) in DMF (1.5 ml) under argon was added 60% sodium hydride dispersion in mineral oil (0.019 g; 0.48 mmol) in one portion. After a further 15 minutes a solution of 2-bromo-1-(3,4-difluorophenyl)ethanone (0.094 g; 0.40 mmol) was added and the resultant solution stirred for 4 hours. Brine (30 ml) was added and the mixture stirred for a further 0.5 hours then allowed to stand at room temperature. The precipitate was filtered, washed with water and air dried. The solid was dissolved in DCM/MeOH/DMSO (10/3/2)(15 ml) and concentrated to approximately 1-2ml. The residual solution was purified by MDAP to afford the title compound (0.043 g; 24%). ¹H NMR (CDCl₃) δ: 1.20-1.34 (1H, m), 1.47-1.51 (2H, d), 1.72-1.82 (4H, m), 1.88-1.92 (1H, d), 1.99-2.11 (2H, m), 4.80 (2H, s), 7.27-7.35(1H, m), 7.71 -7.74 (2H, m), 7.80-7.90 (2H, m), 8.60-8.70 (2H, m). Mass Spectrum (Electrospray LC/MS) Found 451 (MH⁺). C₂₃H₁₉F₅N₂O₂ requires 450. Ret. time 3.91 min.

The compounds in the table below were prepared using similar methods to those described for the Examples above. Method: A=sodium hydride as base (using method similar to that in Example 1). Method B=method similar to that in Example 32. Method C=method similar to that in Example 51. Work-up and purification was carried out using appropriate methods similar to those described in the examples above.

Phenacyl halides were obtained commercially or synthesised by literature methods and arylglycine starting materials were obtained commercially.

| | | | Mass spectrum | |
|---|---|---|---|---|
| | | | (Electrospray | |
| | | | LC/MS), API⁺ | |
| Ex | Structure | Method | Ret. time (min) | Name |
| 4 | 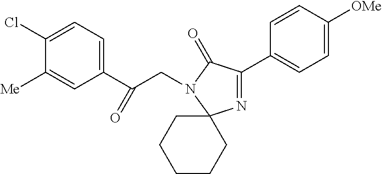 | A | Found 425 (MH⁺). C₂₄H₂₅³⁵ClN₂O₃ requires 424. 3.88. | 1-[2-(4-chloro-3-methylphenyl)- 2-oxoethyl]-3-[4- (methyloxy)phenyl]-1,4- diazaspiro[4.5]dec-3-en-2-one |
| 5 | 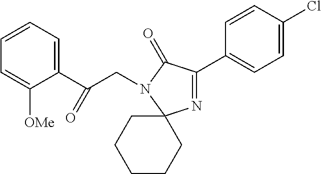 | A | Found 411 (MH⁺). C₂₃H₂₃³⁵ClN₂O₃ requires 410. 3.86. | 3-(4-chlorophenyl)-1-{2-[2- (methyloxy)phenyl]-2-oxoethyl}- 1,4-diazaspiro[4.5]dec-3-en-2- one |
| 6 | 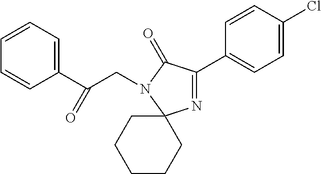 | A | Found 381 (MH⁺). C₂₂H₂₁³⁵ClN₂O₂ requires 380. 3.80. | 3-(4-chlorophenyl)-1-(2-oxo-2- phenylethyl)-1,4- diazaspiro[4.5]dec-3-en-2-one |
| 7 | 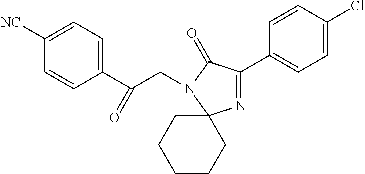 | A | Found 406 (MH⁺). C₂₃H₂₀³⁵ClN₃O₂ requires 405. 3.72. | 4-{[3-(4-chlorophenyl)-2-oxo- 1,4-diazaspiro[4.5]dec-3-en-1- yl]acetyl}benzonitrile |
| 8 | 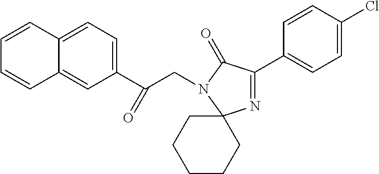 | A | Found 431 (MH⁺). C₂₆H₂₃³⁵ClN₂O₂ requires 430. 4.09. | 3-(4-chlorophenyl)-1-[2-(2- naphthalenyl)-2-oxoethyl]-1,4- diazaspiro[4.5]dec-3-en-2-one |
| 9 | 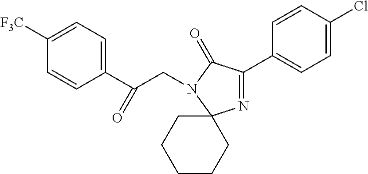 | A | Found 449 (MH⁺). C₂₃H₂₀³⁵ClF₃N₂O₂ requires 448. 4.01. | 3-(4-chlorophenyl)-1-{2-oxo-2- [4-(trifluoromethyl)phenyl]ethyl}- 1,4-diazaspiro[4.5]dec-3-en-2- one |
| 10 | 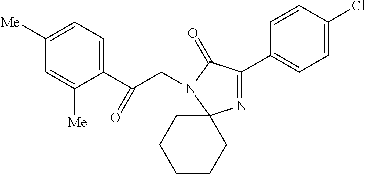 | A | Found 409 (MH⁺). C₂₄H₂₅³⁵ClN₂O₂ requires 408. 4.10. | 3-(4-chlorophenyl)-1-[2-(2,4- dimethylphenyl)-2-oxoethyl]-1,4- diazaspiro[4.5]dec-3-en-2-one |
| 11 | 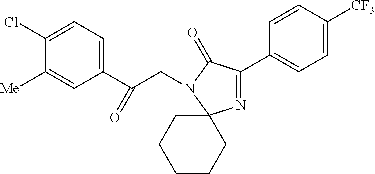 | A | Found 463 (MH⁺). C₂₄H₂₂³⁵ClF₃N₂O₂ requires 462. Ret. time 4.06 min | 1-[2-(4-chloro-3-methylphenyl)- 2-oxoethyl]-3-[4- (trifluoromethyl)phenyl]-1,4- diazaspiro[4.5]dec-3-en-2-one |
| 12 | 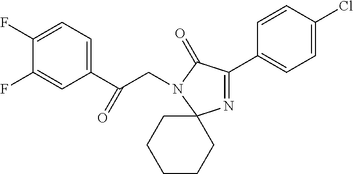 | A | Found 417 (MH⁺). C₂₂H₁₉³⁵ClF₂N₂O₂ requires 416. 3.88. | 3-(4-chlorophenyl)-1-[2-(3,4- difluorophenyl)-2-oxoethyl]-1,4- diazaspiro[4.5]dec-3-en-2-one |
| 13 | 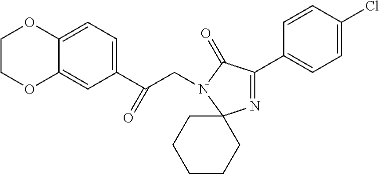 | A | Found 439 (MH⁺). C₂₄H₂₃³⁵ClN₂O₄ requires 438. 3.82. | 3-(4-chlorophenyl)-1-[2-(2,3- dihydro-1,4-benzodioxin-6-yl)-2- oxoethyl]-1,4- diazaspiro[4.5]dec-3-en-2-one |
| 14 | 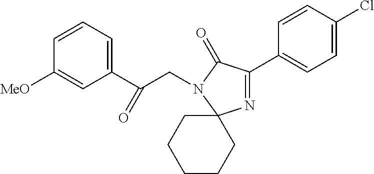 | A | Found 411 (MH⁺). C₂₃H₂₃³⁵ClN₂O₃ requires 410. 3.85. | 3-(4-chlorophenyl)-1-{2-[3- (methyloxy)phenyl]-2-oxoethyl}- 1,4-diazaspiro[4.5]dec-3-en-2- one |
| 15 | 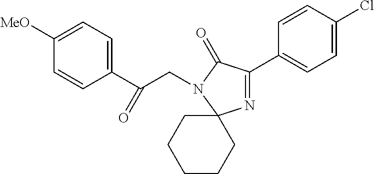 | A | Found 411 (MH⁺). C₂₃H₂₃³⁵ClN₂O₃ requires 410. 3.79. | 3-(4-chlorophenyl)-1-{2-[4- (methyloxy)phenyl]-2-oxoethyl}- 1,4-diazaspiro[4.5]dec-3-en-2- one |
| 16 | 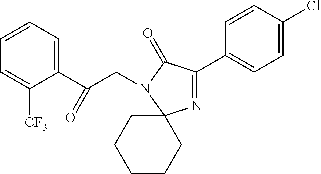 | A | Found 449 (MH⁺). C₂₃H₂₀³⁵ClF₃N₂O₂ requires 448. 3.94. | 3-(4-chlorophenyl)-1-{2-oxo-2- [2-(trifluoromethyl)phenyl]ethyl}- 1,4-diazaspiro[4.5]dec-3-en-2- one |
| 17 | 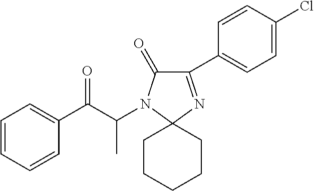 | A | Found 395 (MH⁺). C₂₃H₂₃³⁵ClN₂O₂ requires 394. 3.88. | 3-(4-chlorophenyl)-1-(1-methyl- 2-oxo-2-phenylethyl)-1,4- diazaspiro[4.5]dec-3-en-2-one |
| 18 | 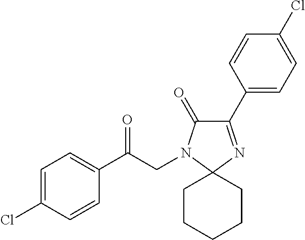 | A | Found 415 (MH⁺). C₂₂H₂₀³⁵Cl₂N₂O₂ requires 414. 3.87. | 3-(4-chlorophenyl)-1-[2-(4- chlorophenyl)-2-oxoethyl]-1,4- diazaspiro[4.5]dec-3-en-2-one |
| 19 | 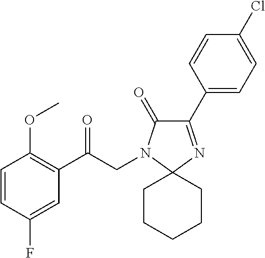 | A | Found 429 (MH⁺). C₂₃H₂₂³⁵ClFN₂O₃ Requires 428. 3.85. | 3-(4-chlorophenyl)-1-{2-[5-fluoro- 2-(methyloxy)phenyl]-2- oxoethyl}-1,4-diazaspiro[4.5]dec- 3-en-2-one |
| 20 | 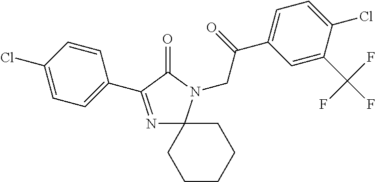 | A | Found 483 (MH⁺). C₂₃H₁₉³⁵Cl₂F₃N ₂O₂ Requires 482. 4.19 | 3-(4-chlorophenyl)-1-{2-[4- chloro-3-(trifluoromethyl)phenyl]- 2-oxoethyl}-1,4- diazaspiro[4.5]dec-3-en-2-one |
| 21 | 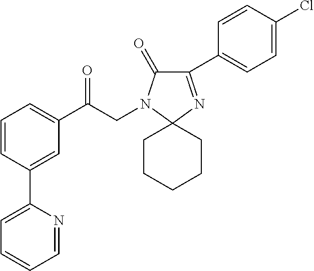 | A | Found 458 (MH⁺). C₂₇H₂₄³⁵ClN₃O₂ Requires 457. 3.72 | 3-(4-chlorophenyl)-1-{2-oxo-2-[3- (2-pyridinyl)phenyl]ethyl}-1,4- diazaspiro[4.5]dec-3-en-2-one |
| 22 | 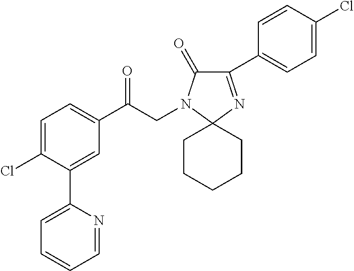 | A | Found 492 (MH⁺). C₂₇H₂₃³⁵Cl₂N₃O₂ Requires 491. 3.78 | 3-(4-chlorophenyl)-1-{2-[4- chloro-3-(2-pyridinyl)phenyl]-2- oxoethyl}-1,4-diazaspiro[4.5]dec- 3-en-2-one |
| 23 | 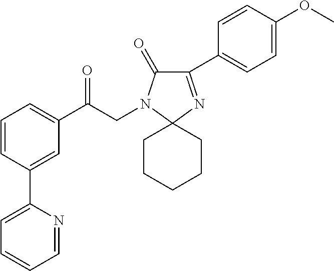 | A | Found 454 (MH⁺). C₂₈H₂₇N₃O₂ Requires 453. 3.43 | 3-[4-(methyloxy)phenyl]-1-{2- oxo-2-[3-(2- pyridinyl)phenyl]ethyl}-1,4- diazaspiro[4.5]dec-3-en-2-one |
| 24 | 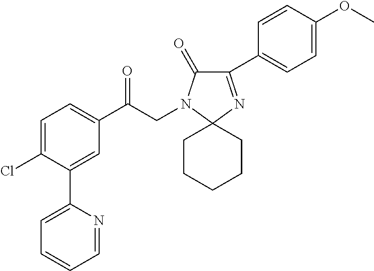 | A | Found 488 (MH⁺). C₂₈H₂₆³⁵ClN₃O₃ Requires 487. 3.51 | 1-{2-[4-chloro-3-(2- pyridinyl)phenyl]-2-oxoethyl}-3- [4-(methyloxy)phenyl]-1,4- diazaspiro[4.5]dec-3-en-2-one |
| 25 | 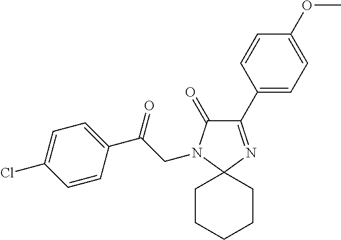 | A | Found 411 (MH⁺). C₂₃H₂₃³⁵ClN₂O₃ Requires 410. 3.62 | 1-[2-(4-chlorophenyl)-2- oxoethyl]-3-[4- (methyloxy)phenyl]-1,4- diazaspiro[4.5]dec-3-en-2-one |
| 26 | 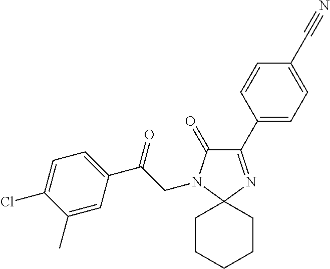 | A | Found 420 (MH⁺). C₂₄H₂₂³⁵ClN₃O₂ Requires 419. 3.74 | 4-{4-[2-(4-chloro-3- methylphenyl)-2-oxoethyl]-3-oxo- 1,4-diazaspiro[4.5]dec-1-en-2- yl}benzonitrile |
| 27 | 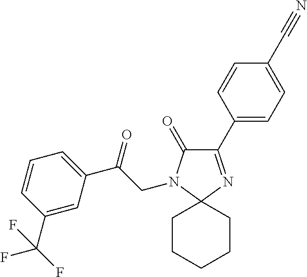 | A | Found 440 (MH⁺). C₂₄H₂₀³⁵F₃N₃O₂ Requires 439. 3.64 | 4-(3-oxo-4-{2-oxo-2-[3- (trifluoromethyl)phenyl]ethyl}- 1,4-diazaspiro[4.5]dec-1-en-2- yl)benzonitrile |
| 28 | 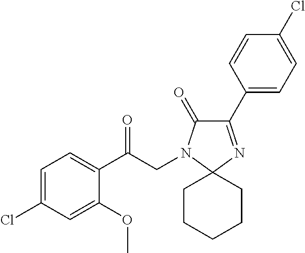 | A | Found 445 (MH⁺). C₂₃H₂₂³⁵Cl₂N₂O₃ Requires 444. 3.93 | 1-{2-[4-chloro-2- (methyloxy)phenyl]-2-oxoethyl}- 3-(4-chlorophenyl)-1,4- diazaspiro[4.5]dec-3-en-2-one |
| 29 | 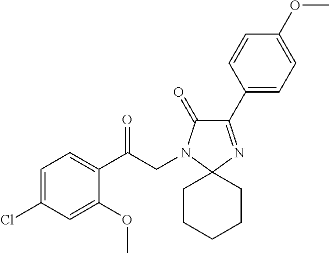 | A | Found 441 (MH⁺). C₂₄H₂₅³⁵ClN₂O₄ Requires 440. 3.71 | 1-{2-[4-chloro-2- (methyloxy)phenyl]-2-oxoethyl}- 3-[4-(methyloxy)phenyl]-1,4- diazaspiro[4.5]dec-3-en-2-one |
| 30 | 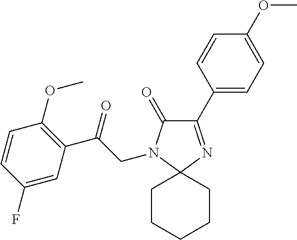 | A | Found 425 (MH⁺). C₂₄H₂₅FN₂O₄ Requires 424. 3.57 | 1-{2-[5-fluoro-2- (methyloxy)phenyl]-2-oxoethyl}- 3-[4-(methyloxy)phenyl]-1,4- diazaspiro[4.5]dec-3-en-2-one |
| 31 | 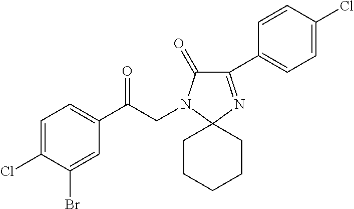 | A | Found 493 (MH⁺). C₂₂H₁₉⁷⁹Br³⁵Cl₂N₂O₂ Requires 492. 4.05 | 1-[2-(3-bromo-4-chlorophenyl)-2- oxoethyl]-3-(4-chlorophenyl)-1,4- diazaspiro[4.5]dec-3-en-2-one |
| 33 | 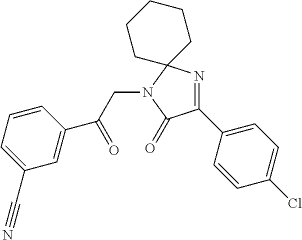 | A | Found 406 (MH⁺). C₂₃H₂₀³⁵ClN₃O₂ Requires 405. 3.61 | 3-{[3-(4-chlorophenyl)-2-oxo-1,4- diazaspiro[4.5]dec-3-en-1- yl]acetyl}benzonitrile |
| 34 | 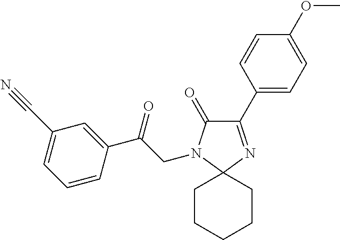 | A | Found 402 (MH⁺). C₂₄H₂₃N₃O₃ Requires 401. 3.31 | 3-({3-[4-(methyloxy)phenyl]-2- oxo-1,4-diazaspiro[4.5]dec-3-en- 1-yl}acetyl)benzonitrile |
| 35 | 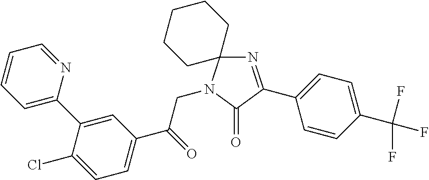 | A | Found 526 (MH⁺). C₂₈H₂₃³⁵ClF₃N₃O₂ Requires 525. 3.80 | 1-{2-[4-chloro-3-(2- pyridinyl)phenyl]-2-oxoethyl}-3- [4-(trifluoromethyl)phenyl]-1,4- diazaspiro[4.5]dec-3-en-2-one |
| 36 | 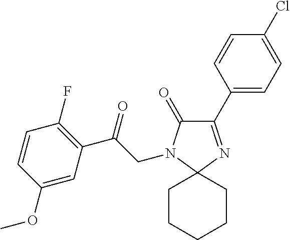 | A | Found 429 (MH⁺). C₂₃H₂₂³⁵ClFN₂O₃ Requires 428. 3.83 | 3-(4-chlorophenyl)-1-{2-[2-fluoro- 5-(methyloxy)phenyl]-2- oxoethyl}-1,4-diazaspiro[4.5]dec- 3-en-2-one |
| 37 | 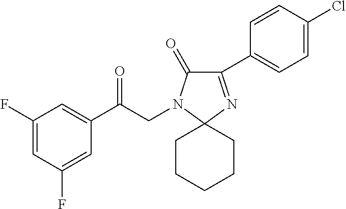 | A | Found 417 (MH⁺). C₂₂H₁₉³⁵ClF₂N₂O₂ Requires 416. 3.80 | 3-(4-chlorophenyl)-1-[2-(3,5- difluorophenyl)-2-oxoethyl]-1,4- diazaspiro[4.5]dec-3-en-2-one |
| 38 | 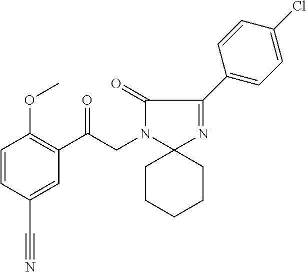 | A | Found 436 (MH⁺). C₂₄H₂₂³⁵ClN₃O₃ Requires 435. 3.78 | 3-{[3-(4-chlorophenyl)-2-oxo-1,4- diazaspiro[4.5]dec-3-en-1- yl]acetyl}-4- (methyloxy)benzonitrile |
| 39 | 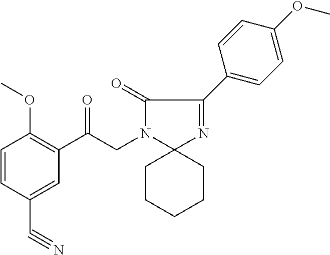 | A | Found 432 (MH⁺). C₂₅H₂₅N₃O₄ Requires 431. 3.51 | 4-(methyloxy)-3-({3-[4- (methyloxy)phenyl]-2-oxo-1,4- diazaspiro[4.5]dec-3-en-1- yl}acetyl)benzonitrile |
| 40 | 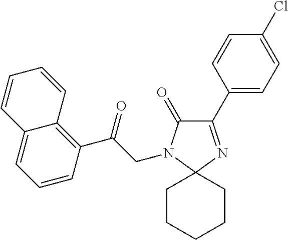 | A | Found 431 (MH⁺). C₂₆H₂₃³⁵ClN₂O₂ Requires 430. 4.12 | 3-(4-chlorophenyl)-1-[2-(1- naphthalenyl)-2-oxoethyl]-1,4- diazaspiro[4.5]dec-3-en-2-one |
| 41 | 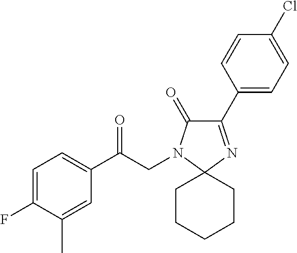 | A | Found 413 (MH⁺). C₂₃H₂₂³⁵ClFN₂O₂ Requires 412. 3.99 | 3-(4-chlorophenyl)-1-[2-(4-fluoro- 3-methylphenyl)-2-oxoethyl]-1,4- diazaspiro[4.5]dec-3-en-2-one |
| 42 | 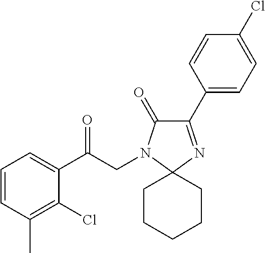 | A | Found 429 (MH⁺). C₂₃H₂₂³⁵Cl₂N₂O₂ Requires 428. 4.14 | 1-[2-(2-chloro-3-methylphenyl)- 2-oxoethyl]-3-(4-chlorophenyl)- 1,4-diazaspiro[4.5]dec-3-en-2- one |
| 43 | 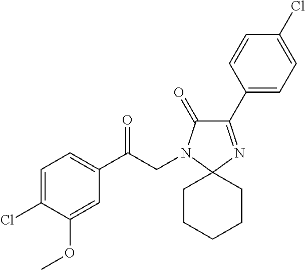 | A | Found 445 (MH⁺). C₂₃H₂₂³⁵Cl₂N₂O₃ Requires 444. 3.80 | 1-{2-[4-chloro-3- (methyloxy)phenyl]-2-oxoethyl}- 3-(4-chlorophenyl)-1,4- diazaspiro[4.5]dec-3-en-2-one |
| 44 | 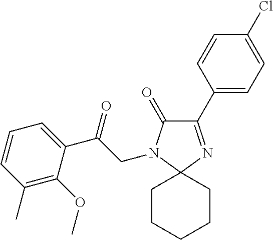 | A | Found 425 (MH⁺). C₂₄H₂₅³⁵ClN₂O₃ Requires 424. 3.85 | 3-(4-chlorophenyl)-1-{2-[3- methyl-2-(methyloxy)phenyl]-2- oxoethyl}-1,4-diazaspiro[4.5]dec- 3-en-2-one |
| 45 | 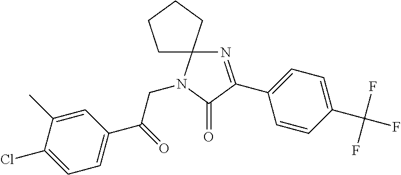 | A | Found 449 (MH⁺). C₂₃H₂₀³⁵ClF₃N₃O2 Requires 448. 3.93 | 1-[2-(4-chloro-3-methylphenyl)- 2-oxoethyl]-3-[4- (trifluoromethyl)phenyl]-1,4- diazaspiro[4.4]non-3-en-2-one |
| 46 | 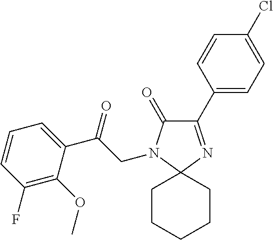 | A | Found 429 (MH⁺). C₂₃H₂₂³⁵ClFN₂O₃ Requires 428. 3.94 | 3-(4-chlorophenyl)-1-{2-[3-fluoro- 2-(methyloxy)phenyl]-2- oxoethyl}-1,4-diazaspiro[4.5]dec- 3-en-2-one |
| 47 | 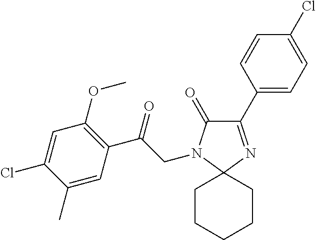 | A | Found 459 (MH⁺). C₂₄H₂₄³⁵Cl₂N₂O₃ Requires 458. 4.20 | 1-{2-[4-chloro-5-methyl-2- (methyloxy)phenyl]-2-oxoethyl}- 3-(4-chlorophenyl)-1,4- diazaspiro[4.5]dec-3-en-2-one |
| 48 | 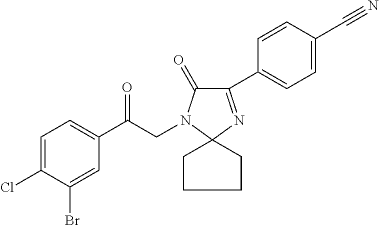 | A | Found 472 (MH⁺). C₂₂H₁₇⁷⁹Br³⁵ClN₃O₂ Requires 471. 3.67 | 4-{4-[2-(3-bromo-4- chlorophenyl)-2-oxoethyl]-3-oxo- 1,4-diazaspiro[4.4]non-1-en-2- yl}benzonitrile |
| 49 | 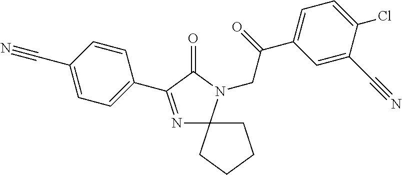 | B | Found 417 (MH⁺). C₂₃H₁₇³⁵ClN₄O₂ Requires 416. 3.28 | 2-chloro-5-{[3-(4-cyanophenyl)- 2-oxo-1,4-diazaspiro[4.4]non-3- en-1-yl]acetyl}benzonitrile |
| 50 | 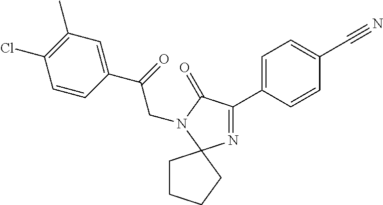 | A | Found 406 (MH⁺). C₂₃H₂₀³⁵ClN₃O₂ Requires 405. 3.47 | 4-{4-[2-(4-chloro-3- methylphenyl)-2-oxoethyl]-3-oxo- 1,4-diazaspiro[4.4]non-1-en-2- yl}benzonitrile |
| 52 | 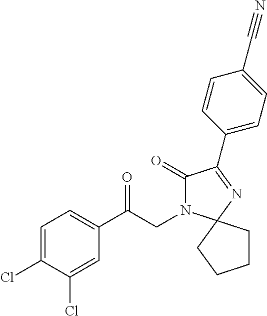 | C | Found 426 (MH⁺). C₂₂H₁₇³⁵Cl₂N₃O₂ Requires 425. 3.52 | 4-{4-[2-(3,4-dichlorophenyl)-2- oxoethyl]-3-oxo-1,4- diazaspiro[4.4]non-1-en-2- yl}benzonitrile |
| 53 | 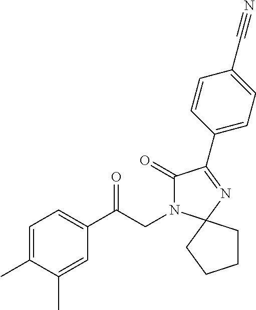 | C | Found 386 (MH⁺). C₂₄H₂₃N₃O₂ requires 385 3.40 | 4-{4-[2-(3,4-dimethylphenyl)-2- oxoethyl]-3-oxo-1,4- diazaspiro[4.4]non-1-en-2- yl}benzonitrile |

### Example 32

2-chloro-5-{[3-(4-chlorophenyl)-2-oxo-1,4-diazaspiro[4.5]dec-3-en-1-yl]acetyl}benzonitrile

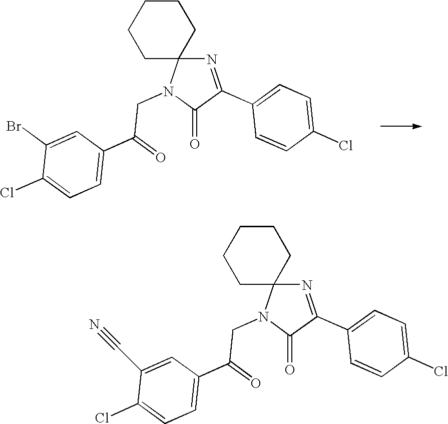

A mixture of 1-[2-(3-bromo-4-chlorophenyl)-2-oxoethyl]-3-(4-chlorophenyl) -1,4-diazaspiro[4.5]dec-3-en-2-one (0.10 g, 0.213 mmol, example 31) and copper (I) cyanide (0.038 g, 0.426 mmol) were combined in 1-methyl-2-pyrrolidinone (10 ml) and heated to vigorous reflux using an isomantle under argon for 1.5 h. The mixture was cooled, poured into saturated sodium hydrogen carbonate (400 ml) and extracted with ethyl acetate (2×150 ml). The combined organics were dried (Na₂SO₄) and solvent evaporated under reduced pressure. The residue was dissolved in DMSO to give a total volume of 1.8 ml, divided into two equal portions and each portion purified by MDAP. The first fraction from each MDAP purification was combined and evaporated under reduced pressure to afford the title compound (0.038 g) as a beige solid. Mass Spectrum (Electrospray LC/MS) Found 440 (MH⁺). C₂₃H₁₉³⁵Cl₂N₃O₂ requires 439. Ret. time 3.87 min

### Example 51

4-(4-{2-[4-chloro-3-(trifluoromethyl)phenyl]-2-oxoethyl}-3-oxo-1,4-diazaspiro[4.4]non-1-en-2-yl)benzonitrile

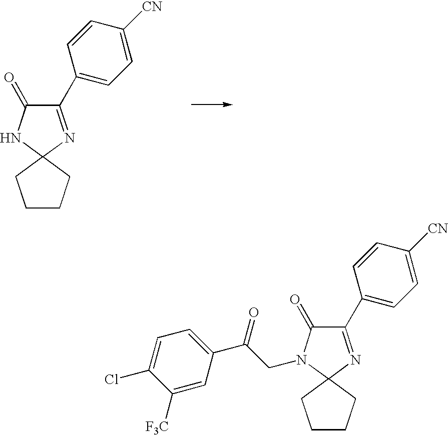

To a solution of 4-(3-oxo-1,4-diazaspiro[4.4]non-1-en-2-yl)benzonitrile D19 (1.0 eq., 0.4184 mmol, 100 mg) in dimethylformamide (2 ml), 60% sodium hydride dispersion in mineral oil (1.1 eq., 18 mg) was added at 0° C. The reaction was stirred for 20 minutes at r.t. 2-Bromo-1-[4-chloro-3-(trifluoromethyl)phenyl]ethanone (1.2 eq., 0.50 mmol, 151 mg) dissolved in dimethylformamide (2 ml) was added by syringe pump over 30 minutes. The reaction was left stirring for 24 hours at r.t. TLC and LCMS showed that 4-(3-oxo-1,4-diazaspiro[4.4]non-1 -en-2-yl)benzonitrile was still present. 2-Bromo-1-[4-chloro-3-(trifluoromethyl)phenyl]ethanone (1.2 eq., 0.50 mmol, 151 mg) and sodium hydride (1.1 eq., 18 mg) were added portionwise. The reaction was stirred for 3 hours at r.t. Water (30 ml) was added and the aqueous layer extracted with ethyl acetate (2×30 ml); organics were alternatively washed with water (2×30 ml) and brine (2×30 ml). The organics were combined, dried over Na₂SO₄, filtered and the solvent was evaporated to afford the crude product. The crude product was purified by column chromatography (ethyl acetate in hexane) on silica column to yield the titled compound, 46 mg, 24%, Found 460 (MH⁺). C₂₃H₁₇³⁵ClF₃N₂O₃ Requires 459. ¹H NMR δ (CDCl₃, 400 MHz, free base) 1.920 ( 6H, m), 2.129 (2H, m), 4.843 (2H, s), 7.705 (1H, d), 7.762 (2H, d), 8.125 (1H, dd), 8.333 (1H, d), 8.588 (2H, d).

1-28. (canceled)

29. A compound of formula (I) or a salt thereof: 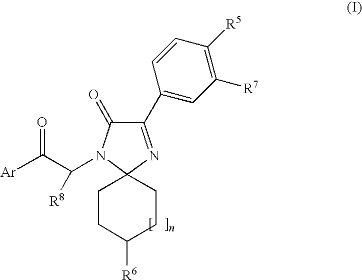
wherein:
Ar is naphthyl optionally substituted with one or more groups Y, pyridinyl optionally substituted with one or more groups Y, or group (A) 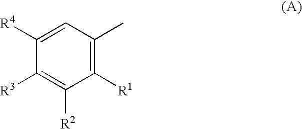
Y is C₁-C₄alkyl, C₁-C₄alkoxy, halo, haloC₁-C₄alkyl, haloC₁-C₄alkoxy, or cyano;
R¹ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H or C₁-C₄alkyl, heteroaryl, or cyano;
R² is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H and C₁-C₄alkyl, heteroaryl, or cyano;
R³ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H or C₁-C₄alkyl, heteroaryl, and cyano;
or R² and R³ together form a group selected from —O—CH₂—O— and —O—CH₂-CH₂—O—;
R⁴ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ (where R⁹ and R¹⁰ are independently H or C₁-C₄alkyl), heteroaryl, or cyano;
R⁵ is H, C₁-C₄alkyl, C₁-C₄alkoxy, halo, haloC₁-C₄alkyl, haloC₁-C₄alkoxy, C₁-C₄alkoxyC₁-C₄alkyl, C₁-C₄alkoxyC₁-C₄alkoxy, or cyano;
R⁷ is H, C₁-C₄alkyl, C₁-C₄alkoxy, halo, haloC₁-C₄alkyl, haloC₁-C₄alkoxy, C₁-C₄alkoxyC₁-C₄alkyl, C₁-C₄alkoxyC₁-C₄alkoxy, or cyano;
R⁶ is H or methyl;
R⁸ is H or methyl; and
n is 0, 1 or 2.

30. A compound as claimed in claim 29 wherein Ar is naphthyl optionally substituted with one or more groups Y, pyridinyl optionally substituted with one or more groups Y, and the group (A).

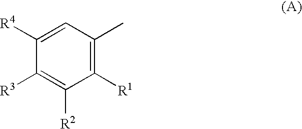

31. A compound as claimed in claim 29 wherein R¹ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H or C₁-C₄alkyl, heteroaryl, or cyano.

32. A compound as claimed in claim 29 wherein:
R² is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H or C₁-C₄alkyl, heteroaryl, or cyano.

33. A compound as claimed in claim 29 wherein:
R³ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H or C₁-C₄alkyl), heteroaryl, or cyano.

34. A compound as claimed in claim 29 wherein R² and R³ together form a group which is —O—CH₂—O— or —O—CH₂-CH₂—O—.

35. A compound as claimed in claim 29 wherein R⁴ is H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR⁹R¹⁰ where R⁹ and R¹⁰ are independently H or C₁-C₄alkyl, heteroaryl, or cyano.

36. A compound as claimed in claim 29 wherein R⁵ is H, C₁-C₄alkyl, C₁-C₄alkoxy, halo, haloC₁-C₄alkyl, haloC₁-C₄alkoxy, C₁-C₄alkoxyC₁-C₄alkyl, C₁-C₄alkoxyC₁-C₄alkoxy, or cyano.

37. A compound as claimed in claim 29 wherein R⁶ is H or methyl.

38. A compound as claimed in claim 29 wherein R⁷ is H, C₁-C₄alkyl, C₁-C₄alkoxy, halo, haloC₁-C₄alkyl, haloC₁-C₄alkoxy, C₁-C₄alkoxyC₁-C₄alkyl, C₁-C₄alkoxyC₁-C₄alkoxy, or cyano.

39. A compound as claimed in claim 29 wherein R⁸ is H or methyl.

40. A compound as claimed in claims 29 wherein n is 0, 1 or 2.

41. A compound as claimed in claim 29 wherein Y is C₁-C₄alkyl, C₁-C₄alkoxy, halo, haloC₁-C₄alkyl, haloC₁-C₄alkoxy, or cyano.

42. A compound as claimed in claim 29 wherein:
Ar is naphthyl or the group (A) 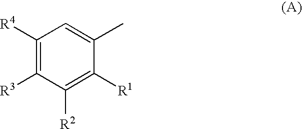
R¹ is H, methyl, methoxy, halo, trifluoromethyl, pyridinyl, or cyano;
R² is H, methyl, methoxy, halo, trifluoromethyl, pyridinyl, and cyano;
R³ is H, C₁-C₂alkyl, C₁-C₂alkoxy, halo, haloC₁-C₂alkyl, haloC₁-C₂alkoxy, pyridinyl, or cyano;
or R² and R³ together form a group which is —O—CH₂—O— and —O—CH₂-CH₂—O—
R⁴ is H, methyl, methoxy, halo, trifluoromethyl, pyridinyl, or cyano;
R⁵ is H, C₁-C₂alkyl, C₁-C₂alkoxy, halo, haloC₁-C₂alkyl, or cyano;
R⁷ is H, C₁-C₂alkyl, C₁-C₂alkoxy, halo, or haloC₁-C₂alkyl;
R⁶ is H;
R⁸ is H or methyl; and
n is 0 or 1

43. A compound as claimed in claim 29 wherein:
Ar naphthyl or the group (A) 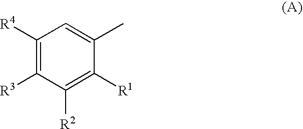
R¹ is H, methyl, methoxy, chloro, fluoro, tritluoromethyl, pyridinyl, or cyano;
R² is H, methyl, methoxy, bromo, chloro, fluoro, trifluoromethyl, pyridinyl, or cyano;
R³ is H, C₁-C₂alkyl, C₁-C₂alkoxy, chloro, fluoro, chloroC₁-C₂alkyl, fluoroC₁-C₂alkyl, chloroC₁-C₂alkoxy, fluoroC₁-C₂alkoxy, pyridinyl, or cyano;
or R² and R³ together form a group which is —O—CH₂—O— or —O—CH₂-CH₂—O—;
R⁴ is H, methyl, methoxy, chloro, fluoro, trifluoromethyl, pyridinyl, or cyano;
R⁵ is H, methyl, methoxy, halo, trifluoromethyl or cyano;
R⁷ is H, C₁-C₂alkyl, C₁-C₂alkoxy, halo, or haloC₁-C₂alkyl;
R⁶ is H;
R⁸ is H or methyl; and
n is 0 or 1.

44. A compound as claimed in claim 29 which is:
1-[2-(4-chloro-3-methylphenyl)-2-oxoethyl]-3-(4-chlorophenyl )-1,4-diazaspiro[4.5]dec-3-en-2-one;
3-(4-chlorophenyl)-1-{2-oxo-2-[3-(trifluoromethyl)phenyl]ethyl}-1,4-diazaspiro[4.5]dec-3-en-2-one;
1-[2-(3,4-difluorophenyl)-2-oxoethyl]-3-[4-(trifluoromethyl)phenyl]-1,4-diazaspiro[4.5]dec-3-en-2-one;
1-[2-(4-chloro-3-methyl phenyl)-2-oxoethyl]-3-[4-(methyloxy)phenyl]-1,4-diazaspiro[4.5]dec-3-en-2-one;
3-(4-chlorophenyl)-1-{2-[2-(methyloxy)phenyl]-2-oxoethyl}-1,4-diazaspiro[4.5]dec-3-en-2-one;
3-(4-chlorophenyl )-1-(2-oxo-2-phenylethyl )-1,4-diazaspiro[4.5]dec-3-en-2-one;
4-{[3-(4-chlorophenyl)-2-oxo-1,4-diazaspiro[4.5]dec-3-en-1-yl]acetyl}benzonitrile;
3-(4-chlorophenyl )-1-[2-(2-naphthalenyl)-2-oxoethyl]-1,4-d iazaspiro[4.5]dec-3-en-2-one;
3-(4-chlorophenyl)-1-{2-oxo-2-[4-(trifluoromethyl)phenyl]ethyl}- 1,4-diazaspiro[4.5]dec-3-en-2-one;
3-(4-chlorophenyl )-1-[2-(2,4-dimethylphenyl)-2-oxoethyl]-1,4-diazaspiro[4.5]dec-3-en-2-one;
1-[2-(4-chloro-3-methylphenyl)-2-oxoethyl]-3-[4-(trifluoromethyl)phenyl]-1,4-diazaspiro[4.5]dec-3-en-2-one;
3-(4-chlorophenyl)-1-[2-(3,4-d ifluorophenyl)-2-oxoethyl]-1,4-diazaspiro[4.5]dec-3-en-2-one;
3-(4-chlorophenyl)-1-[2-(2, 3-dihydro-1,4-benzodioxin-6-yl)-2-oxoethyl]-1,4-diazaspiro[4.5]dec-3-en-2-one;
3-(4-chlorophenyl)-1-{2-[3-(methyloxy)phenyl]-2-oxoethyl}-1,4-diazaspiro[4.5]dec-3-en-2-one;
3-(4-chlorophenyl)-1-{2-[4-(methyloxy)phenyl]-2-oxoethyl}-1,4-diazaspiro[4.5]dec-3-en-2-one;
3-(4-chlorophenyl)-1-{2-oxo-2-[2-(trifl uoromethyl)phenyl]ethyl}-1,4-diazaspiro[4.5]dec-3-en-2-one;
3-(4-chlorophenyl)-1-(1-methyl-2-oxo-2-phenylethyl)-1,4-diazaspiro[4.5]dec-3-en-2-one;
3-(4-chlorophenyl)-1-[2-(4-chlorophenyl)-2-oxoethyl]-1,4-d iazaspiro[4.5]dec-3-en-2-one;
3-(4-chlorophenyl)-1-{2-[5-fluoro-2-(methyloxy)phenyl]-2-oxoethyl}-1,4-diazaspiro[4.5]dec-3-en-2-one;
3-(4-chlorophenyl)-1-{2-[4-chloro-3-(trifluoromethyl)phenyl]-2-oxoethyl}-1,4-diazaspiro[4.5]dec-3-en-2-one;
3-(4-chlorophenyl)-1-{2-oxo-2-[3-(2-pyridinyl)phenyl]ethyl}-1,4-diazaspiro[4.5]dec-3-en-2-one;
3-(4-chlorophenyl)-1-{2-[4-chloro-3-(2-pyridinyl)phenyl]-2-oxoethyl}-1,4-diazaspiro[4.5]dec-3-en-2-one;
3-[4-(methyloxy)phenyl]-1 -{2-oxo-2-[3-(2-pyridinyl)phenyl]ethyl}-1,4-diazaspiro[4.5]dec-3-en-2-one;
1-{2-[4-chloro-3-(2-pyridinyl)phenyl]-2-oxoethyl}-3-[4-(methyloxy)phenyl]-1,4-diazaspiro[4.5]dec-3-en-2-one;
1-[2-(4-chlorophenyl )-2-oxoethyl]-3-[4-(methyloxy)phenyl]-1,4-diazaspiro[4.5]dec-3-en-2-one;
4-{4-[2-(4-chloro-3-methylphenyl)-2-oxoethyl]-3-oxo-1,4-diazaspiro[4 .5]dec-1-en-2-yl}benzonitrile;
4-(3-oxo-4-{2-oxo-2-[3-(trifluoromethyl)phenyl]ethyl}-1,4-d iazaspiro[4.5]dec-1-en-2-yl)benzonitrile;
1-{2-[4-chloro-2-(methyloxy)phenyl]-2-oxoethyl}-3-(4-chlorophenyl)-1,4-diazaspiro[4.5]dec-3-en-2-one;
1-{2-[4-chloro-2-(methyloxy)phenyl]-2-oxoethyl}-3-[4-(methyloxy)phenyl]-1,4-diazaspiro[4.5]dec-3-en-2-one;
1-{2-[5-fluoro-2-(methyloxy)phenyl]-2-oxoethyl}-3-[4-(methyloxy)phenyl]-1,4-diazaspiro[4.5]dec-3-en-2-one;
1-[2-(3-bromo-4-chlorophenyl)-2-oxoethyl]-3-(4-chlorophenyl)-1,4-diazaspiro[4.5]dec-3-en-2-one;
2-chloro-5-{[3-(4-chlorophenyl)-2-oxo-1,4-diazaspiro[4.5]dec-3-en-1-yl]acetyl}benzonitrile;
3-{[3-(4-chlorophenyl)-2-oxo-1,4-diazaspiro[4.5]dec-3-en-1-yl]acetyl}benzonitrile;
3-({3-[4-(methyloxy)phenyl]-2-oxo-1,4-diazaspiro[4.5]dec-3-en-1-yl}acetyl)benzonitrile;
1-{2-[4-ch loro-3-(2-pyridinyl)phenyl]-2-oxoethyl}-3-[4-(trifluoromethyl)phenyl]-1,4-diazaspiro[4.5]dec-3-en-2-one;
3-(4-chlorophenyl)-1-{2-[2-fluoro-5-(methyloxy)phenyl]-2-oxoethyl}-1,4-diazaspiro[4.5]dec-3-en-2-one;
3-(4-chlorophenyl)-1-[2-(3, 5-difluorophenyl)-2-oxoethyl]-1,4-diazaspiro[4.5]dec-3-en-2-one;
3-{[3-(4-chlorophenyl)-2-oxo-1,4-diazaspiro[4.5]dec-3-en-1 -yl]acetyl}-4-(methyloxy)benzonitrile;
4-(methyloxy)-3-({3-[4-(methyloxy)phenyl]-2-oxo-1,4-diazaspiro[4.5]dec-3-en-1-yl}acetyl)benzonitrile;
3-(4-chlorophenyl )-1-[2-(1-naphthalenyl)-2-oxoethyl]-1,4-diazaspiro[4.5]dec-3-en-2-one;
3-(4-chlorophenyl)-1-[2-(4-fluoro-3-methylphenyl)-2-oxoethyl]-1,4-diazaspiro[4.5]dec-3-en-2-one;
1-[2-(2-chloro-3-methylphenyl)-2-oxoethyl]-3-(4-chlorophenyl)-1,4-diazaspiro[4.5]dec-3-en-2-one;
1-{2-[4-chloro-3-(methyloxy)phenyl]-2-oxoethyl}-3-(4-chlorophenyl)-1,4-diazaspiro[4.5]dec-3-en-2-one;
3-(4-chlorophenyl)-1 -{2-[3-methyl-2-(methyloxy)phenyl]-2-oxoethyl}-1,4-diazaspiro[4.5]dec-3-en-2-one;
1-[2-(4-chloro-3-methylphenyl)-2-oxoethyl]-3-[4-(trifluoromethyl)phenyl]-1,4-diazaspiro[4.4]non-3-en-2-one;
3-(4-chlorophenyl)-1-{2-[3-fluoro-2-(methyloxy)phenyl]-2-oxoethyl}-1,4-diazaspiro[4.5]dec-3-en-2-one;
1-{2-[4-chloro-5-methyl-2-(methyloxy)phenyl]-2-oxoethyl}-3-(4-chlorophenyl)-1,4-diazaspiro[4.5]dec-3-en-2-one;
4-{4-[2-(3-bromo-4-chlorophenyl)-2-oxoethyl]-3-oxo-1,4-diazaspiro[4.4]non-1-en-2-yl}benzonitrile;
2-chloro-5-{[3-(4-cyanophenyl)-2-oxo-1,4-diazaspiro[4.4]non-3-en-1-yl]acetyl}benzonitrile;
4-{4-[2-(4-chloro-3-methylphenyl)-2-oxoethyl]-3-oxo-1,4-diazaspiro[4.4]non-1-en-2-yl}benzonitrile;
4-(4-{2-[4-chloro-3-(trifluoromethyl)phenyl]-2-oxoethyl}-3-oxo-1,4-diazaspiro[4.4]non-1-en-2-yl)benzonitrile;
4-{4-[2-(3,4-dichlorophenyl)-2-oxoethyl]-3-oxo-1,4-diazaspiro[4.4]non-1-en-2-yl}benzonitrile; or
4-{4-[2-(3,4-dimethylphenyl)-2-oxoethyl]-3-oxo-1,4-diazaspi ro[4.4]non-1-en-2-yl}benzonitrile;
or a salt .

45. A method of treating schizophrenia, dementia or attention deficit disorder comprising administering an effective amount of a compound of formula (I) according to claim 29 or a salt thereof, neat or admixed with a pharmaceutically acceptable carrier.

46. A pharmaceutical composition comprising a compound as claimed in claim 29 or a salt thereof and at least one pharmaceutically acceptable carrier, diluent or excipient.
